(19) United States
(12) Patent Application Publication
(10) Pub. No.: US 2021/0379043 A1
(43) Pub. Date: Dec. 9, 2021

(54) COMBINATION TREATMENT OF LIVER DISORDERS

(71) Applicant: Terns Pharmaceuticals, Inc., Foster City, CA (US)

(72) Inventors: Martijn FENAUX, San Mateo, CA (US); Kevin KLUCHER, Bothell, WA (US); Christopher T. JONES, Foster City, CA (US); Thorsten A. KIRSCHBERG, Foster City, CA (US)

(21) Appl. No.: 17/318,994

(22) Filed: May 12, 2021

Related U.S. Application Data
(60) Provisional application No. 63/024,360, filed on May 13, 2020.

(51) Int. Cl. A61K 31/454; A61K 31/53; A61K 31/4439; A61K 31/4748; A61K 31/575; A61P 1/16

(52) U.S. Cl. CPC A61K 31/454; A61K 31/53; A61P 1/16; A61K 31/4748; A61K 31/575; A61K 31/4439

(57) ABSTRACT

Provided herein are methods for treating liver disorders, including non-alcoholic steatohepatitis, and symptoms and manifestations thereof, in a patient which utilize, among others, a combination treatment of an FXR agonist and a THRβ agonist.

### CROSS REFERENCE TO RELATED APPLICATIONS

This application claims priority benefit of U.S. Provisional Application No. 63/024,360, filed May 13, 2020, which is incorporated herein by reference in its entirety.

### FIELD OF THE INVENTION

This invention relates to methods and compositions for treating liver disorder in a patient.

### BACKGROUND

Fatty liver disease (FLD) encompasses a spectrum of disease states characterized by excessive accumulation of fat in the liver often accompanied with inflammation. FLD can lead to non-alcoholic fatty liver disease (NAFLD), which may be characterized by insulin resistance. If untreated, NAFLD can progress to a persistent inflammatory response or non-alcoholic steatohepatitis (NASH), progressive liver fibrosis, and eventually to cirrhosis. In Europe and the US, NAFLD is the second most common reason for liver transplantation. Accordingly, the need for treatment is urgent, but due to the lack of obvious symptoms to the patient, patients may lack the motivation to maintain treatment regimens, particularly burdensome treatment regimens, such as injected medicines, medications that are administered many times a day, or any that produce dangerous or irritating side effects. There is currently no approved treatment of NASH.

### BRIEF SUMMARY

Provided herein are methods and compositions for treating a liver disorder in a patient in need thereof. The methods comprise administering to the patient a Farnesoid X Receptor (FXR) agonist and a thyroid hormone receptor beta (THRβ) agonist.

In one aspect, the disclosure provides methods of reducing hepatic inflammation in a patient in need thereof, comprising administering to the patient a therapeutically effective amount of a FXR agonist and a therapeutically effective amount of a THRβ agonist. The administration of a combination of a FXR agonist and a THRβ agonist reduces hepatic inflammation in a patient in need thereof to a significantly greater extent than administration of either agonist by itself. The reduction of hepatic inflammation is characterized by reduced expression of inflammatory genes and markers of leukocyte activation in the liver. In some embodiments, hepatic inflammation is reduced without increasing the low-density lipoprotein cholesterol (LDL-C) levels in the blood of the patient.

In another aspect, the disclosure provides methods of treating a disease or condition characterized by fibrosis of the liver, comprising administering to the patient a therapeutically effective amount of a FXR agonist and a therapeutically effective amount of a THRβ agonist. The administration of a combination of a FXR agonist and a THRβ agonist reduces fibrosis in a patient in need thereof to a significantly greater extent than administration of either agonist alone. The reduction of fibrosis is characterized by histological improvement and reduced expression of pro-fibrotic genes in the liver. In some embodiments, hepatic fibrosis is reduced without increasing the low-density lipoprotein cholesterol (LDL-C) levels in the blood of the patient. In some embodiments, administration of the FXR agonist and the THRβ agonist results in reduction of liver fibrosis and hepatic inflammation.

As set forth herein, the synergy observed when administering the combination of a FXR agonist and a THRβ agonist to patients in need thereof allows for the reduction of the dose of either or both the FXR agonist and the THRβ agonist relative to when either agonist is administered as a monotherapy. The lower doses of the FXR agonist and the THRβ agonist results in an improved therapeutic index and alleviates side effects that are sometimes accompanied with FXR agonism or THRβ agonism.

In some embodiments, the administration of the FXR agonist and the THRβ agonist does not result in pruritus in the patient at a severity of Grade 2 or more. In some embodiments, the administration of the FXR agonist and the THRβ agonist does not result in pruritus of Grade 1 or more. In some embodiments, the administration of the FXR agonist and the THRβ agonist does not result in pruritus.

In another aspect, the disclosure provide methods of treating or preventing NASH in a patient in need thereof, said method comprising administering to the patient a therapeutically effective amount of a FXR agonist and a therapeutically effective amount of a THRβ agonist. In one embodiment, the patient in need thereof is a patient that suffers from fatty liver disease such as NAFLD. In another embodiment, the patient in need thereof is a patient that suffers from metabolic syndrome.

In some embodiments, the FXR agonist and the THRβ agonist are administered simultaneously. In some such embodiments, the FXR agonist and the THRβ agonist are provided as a fixed-dose composition in a single pharmaceutical composition as set forth herein. In other embodiments, the FXR agonist and the THRβ agonist are administered sequentially. In some embodiments, either or both of the FXR agonist and the THRβ agonist are administered orally.

In some embodiments, the patient has a liver disorder and diabetes mellitus. In some embodiments, the patient has a liver disorder and a cardiovascular disorder. In some embodiments, the treatment period is the remaining lifespan of the patient. In some embodiments, the method does not comprise administering an antihistamine, an immunosuppressant, a steroid, rifampicin, an opioid antagonist, or a selective serotonin reuptake inhibitor (SSRI).

In some embodiments, the FXR agonist is administered once daily. In some embodiments, the FXR agonist is administered twice daily. In some embodiments, the THRβ agonist is administered once daily. In some embodiments, the THRβ agonist is administered twice daily. In some embodiments, the administration comprises administering the FXR agonist daily for a treatment period of one or more weeks. In some embodiments, the administration comprises administering the THRβ agonist daily for a treatment period of one or more weeks. In some embodiments, the administration comprises administering the FXR agonist daily and the THRβ agonist daily for a treatment period of one or more weeks.

A variety of different FXR agonists and THRβ agonist can be used to achieve the beneficial effects observed on liver disease as discussed herein. For instance, in some embodiments, the FXR agonist administered to the patient in need thereof is obeticholic acid. In some embodiments, the FXR agonist administered to the patient in need thereof is cilofexor. In some embodiments, the FXR agonist administered to the patient in need thereof is tropifexor. In some embodiments, the FXR agonist administered to the patient in need thereof is EYP001 (Vonafexor, proposed INN). In some embodiments, the FXR agonist administered to the patient in need thereof is MET642 (Metacrine). In some embodiments, the FXR agonist administered to the patient in need thereof is MET409 (Metacrine). In some embodiments, the FXR agonist is EDP-305 (by Enanta). In some embodiments, the FXR agonist is EDP-297 (by Enanta).

In some embodiments, the FXR agonist administered to the patient in need thereof is a compound of formula (I):

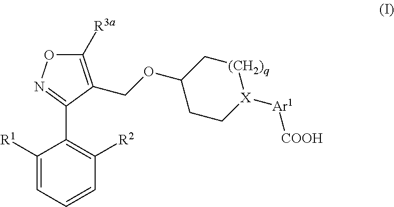

wherein:
q is 1 or 2;
R¹ is chloro, fluoro, or trifluoromethoxy;
R² is hydrogen, chloro, fluoro, or trifluoromethoxy;
R^{3a} is trifluoromethyl, cyclopropyl, or isopropyl;
X is CH or N,
provided that when X is CH, q is 1; and
Ar¹ is indolyl, benzothienyl, naphthyl, phenyl, benzoisothiazolyl, indazolyl, or pyridinyl, each of which is optionally substituted with methyl or phenyl,
or a pharmaceutically acceptable salt thereof.

In some embodiments, the FXR agonist administered to the patient in need thereof is a compound of formula (I) wherein R¹ is chloro or trifluoromethoxy. In some embodiments, the FXR agonist is a compound of formula (I) wherein R² is hydrogen or chloro. In some embodiments, the FXR agonist is a compound of formula (I) wherein R^{3a} is cyclopropyl or isopropyl. In some embodiments, the FXR agonist is a compound of formula (I) wherein Ar¹ is 5-benzothienyl, 6-benzothienyl, 5-indolyl, 6-indolyl, or 4-phenyl, each of which is optionally substituted with methyl. In some embodiments, the FXR agonist is a compound of formula (I) wherein q is 1 and X is N.

In some embodiments, the FXR agonist is

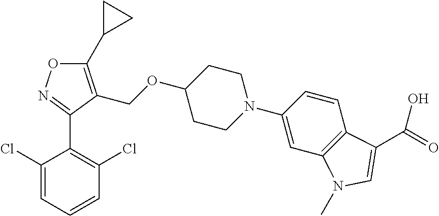 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof.

In some embodiments, the THRβ agonist administered to the patient in need thereof is resmetirom (MGL-3196). In some embodiments, the THRβ agonist is administered to the patient in need thereof VK2809 (by Viking Therapeutics). In some embodiments, the THRβ agonist administered to the patient in need thereof is sobetirome. In some embodiments, the THRβ agonist administered to the patient in need thereof is eprotirome. In some embodiments, the THRβ agonist administered to the patient in need thereof is ALG-055009 (by Aligo). In some embodiments, the THRβ agonist administered to the patient in need thereof is CNPT-101101. In some embodiments, the THRβ agonist administered to the patient in need thereof is CNPT-101207. In some embodiments, the THRβ agonist administered to the patient in need thereof is ASC41 (by Ascletis).

In some embodiments, the THRβ agonist is a compound of Formula (II)

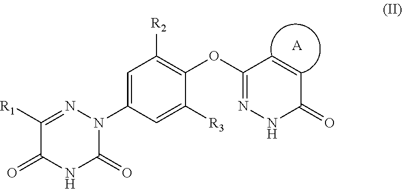

wherein:
R₁ is selected from the group consisting of hydrogen, cyano, substituted or unsubstituted C₁₋₆ alkyl, and substituted or unsubstituted C₃₋₆ cycloalkyl, the substituent being selected from the group consisting of halogen atoms, hydroxy, and C₁₋₆ alkoxy;
R₂ and R₃ are each independently selected from the group consisting of halogen atoms and substituted or unsubstituted C₁₋₆ alkyl, the substituent being selected from the group consisting of halogen atoms, hydroxy, and C₁₋₆ alkoxy;
ring A is a substituted or unsubstituted saturated or unsaturated C₅₋₁₀ aliphatic ring, or a substituted or unsubstituted C₅₋₁₀ aromatic ring, the substituent being one or more substances selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy or C₃₋₆ cycloalkyl, and when two substituents are contained, the two substituents can form a ring structure together with the carbon connected thereto; and
the halogen atoms are selected from the group consisting of F, Cl and Br,
or a pharmaceutically acceptable salt thereof.

In some embodiments, the THRβ agonist administered to the patient in need thereof is a compound of Formula (IIa)

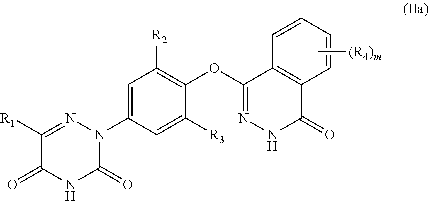

wherein:
R₁ to R₃ are defined as detailed herein for Formula (II);
R₄ is selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl;
m is an integer from the range 1 to 4; and
the halogen atoms are selected from the group consisting of F, Cl and Br.
or a pharmaceutically acceptable salt thereof.

In some embodiments, wherein R₄ is selected from the group consisting of hydrogen, halogen atoms, hydroxy, OCF₃, C₁₋₆ alkyl, C₁₋₆ alkoxy and C₃₋₆ cycloalkyl; and m is an integer from the range 1 to 3.

In some embodiments, wherein R₁ is selected from the group consisting of hydrogen, cyano, and substituted or unsubstituted C₁₋₆ alkyl, the substituent being selected from the group consisting of halogen atoms, hydroxy, and C₁₋₆ alkoxy; and the halogen atoms are selected from the group consisting of F, Cl and Br.

In some embodiments, the THRβ agonist is

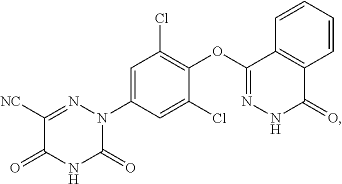 [SMILES: N#Cc1nn(-c2cc(Cl)c(Oc3n[nH]c(=O)c4ccccc34)c(Cl)c2)c(=O)[nH]c1=O]

or a pharmaceutically acceptable salt thereof.

In some embodiments, provided are methods of treating a liver disorder in a patient in need thereof with a Farnesoid X Receptor (FXR) agonist and a thyroid hormone receptor beta (THRβ) agonist, comprising administering a therapeutically effective amount of the FXR agonist, wherein the FXR agonist is

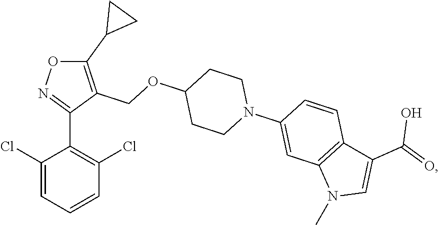 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, and administering a therapeutically effective amount of the THRβ agonist, wherein the THRβ agonist is

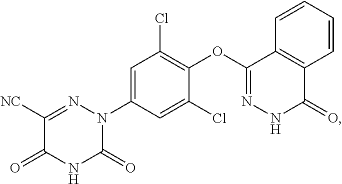 [SMILES: N#Cc1nn(-c2cc(Cl)c(Oc3n[nH]c(=O)c4ccccc34)c(Cl)c2)c(=O)[nH]c1=O]

or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

### BRIEF DESCRIPTION OF THE FIGURES

FIG. 1A shows plasma concentrations of Compound 1 at various time points after intravenous (IV) administration to rats (1 mg/kg), dogs (1 mg/kg) and monkeys (0.3 mg/kg).

FIG. 1B shows plasma concentrations of Compound 1 at various time points after oral administration to mice (10 mg/kg), rats (10 mg/kg), dogs (3 mg/kg) and monkeys (5 mg/kg).

FIG. 2A shows the liver to plasma ratio of the concentration of Compound 1, obeticholic acid (OCA), cilofexor, or tropifexor after 2 mg/kg IV administration to Sprague-Dawley (SD) rats.

FIG. 2B shows the tissue to plasma ratio of the concentration of Compound 1 for kidney, lung, and liver after 2 mg/kg IV administration of Compound 1 to SD rats with or without co-administration of rifampicin.

FIG. 3 shows the tissue distribution of radiolabeled Compound 1 in plasma, liver, small intestine, cecum, kidney, lungs, heart, and skin after 5 mg/kg oral administration of Compound 1 to Long-Evans rats.

FIG. 4 shows the pharmacodynamics of Compound 1 administration, as measured by 7-alpha-hydroxy-4-cholesten-3-one (7AC4), after administration of 0.3 mg/kg, 1 mg/kg or 5 mg/kg oral dose to cynomolgus monkeys.

FIG. 5A shows the pharmacokinetics of Compound 1 administration, after administration of 1 mg/kg oral dose for one day, or 7 consecutive daily doses, to cynomolgus monkeys.

FIG. 5B shows the pharmacodynamics of Compound 1 administration, as measured by 7-alpha-hydroxy-4-cholesten-3-one (7AC4), after administration of 1 mg/kg oral dose for one day, or 7 consecutive daily doses, to cynomolgus monkeys.

FIG. 6 shows RT-qPCR results measuring liver SHP1, liver OSTb, ileum SHP1, and ileum FGF15 RNA expression after administering 10 mg/kg Compound 1, 30 mg/kg OCA, or vehicle control to C5BL/6 mice.

FIG. 7A shows the number of differentially expressed genes (vs. vehicle-treated: fold-change >1.5-fold; p<0.05) modulated by the administration of 10 mg/kg Compound 1 (500 total genes modulated) or 30 mg/kg OCA to C57BL/6 mice (44 total genes modulated), as well as the shared number of differentially expressed genes that are modulated by both compounds (37 total genes).

FIG. 7B shows average expression levels (as shown by CPM value) of select FXR-related genes in C57BL/6 mice treated with 10 mg/kg Compound 1 or 30 mg/kg OCA, or a vehicle control.

FIG. 7C shows the number of pathways enriched (p<0.05) by the administration of 10 mg/kg Compound 1 (32 pathways) or 30 mg/kg OCA to C57BL/6 mice (6 pathways), as well as the number of enriched pathways by either compound (2 pathways).

FIG. 7D shows the 25 pathways most statistically enriched upon administration of 10 mg/kg Compound 1 to C57BL/6 mice, and compares the enrichment of those pathways to the enrichment upon administration of 30 mg/kg OCA.

FIG. 8 shows the design of a study testing the efficacy of Compound 1 on a mouse model of NASH.

FIG. 9 shows the NAFLD Activity Score (NAS) of control mice and mice treated with 10, 30, and 100 mg/kg Compound 1.

FIG. 10A shows the steatosis score of control mice and NASH mice treated with 10, 30, and 100 mg/kg Compound 1.

FIG. 10B shows the inflammation score of control mice and NASH mice treated with 10, 30, and 100 mg/kg Compound 1.

FIG. 10C shows the ballooning score of control mice and NASH mice treated with 10, 30, and 100 mg/kg Compound 1.

FIG. 11A shows a histological section of fibrosis in control mice and NASH mice treated with 100 mg/kg Compound 1.

FIG. 11B shows the amount of fibrosis in control mice and NASH mice treated with 10, 30, and 100 mg/kg Compound 1.

FIG. 12A shows the serum alanine amino transferase (ALT) levels of control mice and NASH mice treated with 10, 30, and 100 mg/kg Compound 1.

FIG. 12B shows aspartate amino transferase (AST) of control mice and NASH mice treated with 10, 30, and 100 mg/kg Compound 1.

FIG. 12C shows serum triglyceride levels of control mice and NASH mice treated with 10, 30, and 100 mg/kg Compound 1.

FIG. 12D shows serum total cholesterol levels of control mice and NASH mice treated with 10, 30, and 100 mg/kg Compound 1.

FIG. 13A shows liver triglyceride levels of control mice and NASH mice treated with 10, 30, and 100 mg/kg Compound 1.

FIG. 13B shows representative histology of steatosis assessment for control mice and NASH mice treated with 100 mg/kg Compound 1.

FIG. 14A shows COL1A1 expression in the liver in control mice and NASH mice treated with 10, 30, and 100 mg/kg Compound 1.

FIG. 14B shows expression levels of inflammatory genes in control mice and NASH mice treated with 30 mg/kg Compound 1.

FIG. 14 C shows expression of fibrosis genes in control mice and NASH mice treated with 30 mg/kg Compound 1.

FIG. 15A shows the effect of Compound 2 on serum cholesterol in rat hypercholesterolemic model.

FIG. 15B shows the effect of Compound 2 on serum triglycerides in rat hypercholesterolemic model.

FIG. 16 shows the effects of Compound 2 on body and organ weight in mouse NASH model.

FIG. 17 shows the effects of Compound 2 on liver steatosis, inflammation, and fibrosis in mouse NASH model.

FIG. 18 shows the effects of Compound 2 on lipids and indicators of liver injury (ALT) in mouse NASH model.

FIG. 19 shows the effects of Compound 2 on expression of genes associated with collagen extracellular matrix and hepatic stellate cell activation.

FIG. 20 shows differential gene expression analysis of select biological processes in a mouse model of NASH treated with 3 mg/kg Compound 1 and/or 1 mg/kg Compound 2.

FIG. 21 shows the number and overlap of differentially expressed genes (DEGs) identified in a mouse model of NASH treated with 3 mg/kg Compound 1, 1 mg/kg Compound 2, or 3 mg/kg Compound 1 and 1 mg/kg Compound 2, relative to a vehicle NASH control.

FIG. 22 shows the number and overlap of biological processes that were significantly enriched in a mouse model of NASH treated with 3 mg/kg Compound 1, 1 mg/kg Compound 2, or 3 mg/kg Compound 1 and 1 mg/kg Compound 2, relative to a vehicle NASH control.

FIG. 23 shows liver steatosis, inflammation, and fibrosis, as well as serum triglyceride, total cholesterol, and alanine aminotransferase (ALT) in a mouse model of NASH treated with 3 mg/kg Compound 1, 1 mg/kg Compound 2, or 3 mg/kg Compound 1 and 1 mg/kg Compound 2, relative to a vehicle NASH control.

FIG. 24 shows expression levels of genes associated with FXR and THRβ pathways in a mouse model of NASH treated with 3 mg/kg Compound 1, 1 mg/kg Compound 2, or 3 mg/kg Compound 1 and 1 mg/kg Compound 2, relative to a vehicle NASH control.

FIG. 25 shows mean expression levels (count per million reads, CPM) of genes associated with fibrosis and inflammation pathways, which were determined by RNAseq. *p<0.05, **p<0.01, ***p<0.001, ****p<0.0001 in a mouse model of NASH vs. vehicle (NASH) control.

### DETAILED DESCRIPTION

### Definitions

As used herein, the following definitions shall apply unless otherwise indicated. Further, if any term or symbol used herein is not defined as set forth below, it shall have its ordinary meaning in the art.

“Comprising” is intended to mean that the compositions and methods include the recited elements, but not exclude others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

“Combination therapy” or “combination treatment” refers to the use of two or more drugs or agents in treatment, e.g., the use of a compound of formula (I) or (II) as utilized herein together with another agent useful to treat liver disorders, such as NAFLD, NASH, and symptoms and manifestations of each thereof is a combination therapy. Administration in “combination” refers to the administration of two agents (e.g., a compound of formula (I) or (II) as utilized herein, and another agent) in any manner in which the pharmacological effects of both manifest in the patient at the same time. Thus, administration in combination does not require that a single pharmaceutical composition, the same dosage form, or even the same route of administration be used for administration of both agents or that the two agents be administered at precisely the same time. Both agent can also be formulated in a single pharmaceutically acceptable composition. A non-limiting example of such a single composition is an oral composition or an oral dosage form. For example, and without limitation, it is contemplated that a compound of formula (I) or (II) can be administered in combination therapy with another agent in accordance with the present invention.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Patient” refers to mammals and includes humans and non-human mammals. Examples of patients include, but are not limited to mice, rats, hamsters, guinea pigs, pigs, rabbits, cats, dogs, goats, sheep, cows, and humans. In some embodiments, patient refers to a human.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the likes.

“Therapeutically effective amount” or dose of a compound or a composition refers to that amount of the compound or the composition that results in reduction or inhibition of symptoms or a prolongation of survival in a patient. The results may require multiple doses of the compound or the composition.

“Treatment” or “treating” refers to an approach for obtaining beneficial or desired results including clinical results. For purposes of this invention, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the invention contemplate any one or more of these aspects of treatment.

As used herein, “delaying” development of a disease means to defer, hinder, slow, retard, stabilize and/or postpone development of the disease and/or slowing the progression or altering the underlying disease process and/or course once it has developed. This delay can be of varying lengths of time, depending on the history of the disease and/or individual being treated. As is evident to one skilled in the art, a sufficient or significant delay can, in effect, encompass prevention, in that the individual does not develop clinical symptoms associated with the disease. A method that “delays” development of a disease is a method that reduces probability of disease development in a given time frame and/or reduces extent of the disease in a given time frame, when compared to not using the method, including stabilizing one or more symptoms resulting from the disease.

An individual who is “at risk” of developing a disease may or may not have detectable disease, and may or may not have displayed detectable disease prior to the treatment methods described herein. “At risk” denotes that an individual has one or more so-called risk factors, which are measurable parameters that correlate with development of a disease. An individual having one or more of these risk factors has a higher probability of developing the disease than an individual without these risk factor(s). These risk factors include, but are not limited to, age, sex, race, diet, history of previous disease, presence of precursor disease and genetic (i.e., hereditary) considerations. Compounds may, in some embodiments, be administered to a subject (including a human) who is at risk or has a family history of the disease or condition.

“Stereoisomer” or “stereoisomers” refer to compounds that differ in the stereogenicity of the constituent atoms such as, without limitation, in the chirality of one or more stereocenters or related to the cis or trans configuration of a carbon-carbon or carbon-nitrogen double bond. Stereoisomers include enantiomers and diastereomers.

“Alkyl” refers to monovalent saturated aliphatic hydrocarbyl groups having from 1 to 12 carbon atoms, preferably from 1 to 10 carbon atoms, and more preferably from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

“Alkylene” refers to a divalent saturated aliphatic hydrocarbyl group having from 1 to 12 carbon atoms, preferably from 1 to 10 carbon atoms, and more preferably from 1 to 6 carbon atoms. This term includes, by way of example, linear and branched hydrocarbyl groups such as methylene (—CH₂—), ethylene (—CH₂CH₂— or —CH(Me)-), propylene (—CH₂CH₂CH₂— or —CH(Me)CH₂—, or —CH(Et)-) and the likes.

“Alkenyl” refers to straight or branched monovalent hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers. Cₓ alkenyl refers to an alkenyl group having x number of carbon atoms.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH). Cₓ alkynyl refers to an alkynyl group having x number of carbon atoms.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Aryl” refers to a monovalent aromatic carbocyclic group of from 6 to 14 carbon atoms having a single ring (e.g., phenyl (Ph)) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Cyano” refers to the group —C≡N.

“Cycloalkyl” refers to saturated or unsaturated but nonaromatic cyclic alkyl groups of from 3 to 10 carbon atoms, preferably from 3 to 8 carbon atoms, and more preferably from 3 to 6 carbon atoms, having single or multiple cyclic rings including fused, bridged, and spiro ring systems. Cₓ cycloalkyl refers to a cycloalkyl group having x number of ring carbon atoms. Examples of suitable cycloalkyl groups include, for instance, adamantyl, cyclopropyl, cyclobutyl, cyclopentyl, and cyclooctyl. One or more the rings can be aryl, heteroaryl, or heterocyclic provided that the point of attachment is through the non-aromatic, non-heterocyclic ring saturated carbocyclic ring. “Substituted cycloalkyl” refers to a cycloalkyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Halo” or “halogen” refers to fluoro, chloro, bromo and iodo and preferably is fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include 5 or 6 membered heteroaryls such as pyridinyl, pyrrolyl, thiophenyl, and furanyl. Other preferred heteroaryls include 9 or 10 membered heteroaryls, such as indolyl, quinolinyl, quinolonyl, isoquinolinyl, and isoquinolonyl.

“Heterocycle” or “heterocyclic” or “heterocycloalkyl” or “heterocyclyl” refers to a saturated or partially saturated, but not aromatic, group having from 1 to 10 ring carbon atoms, preferably from 1 to 8 carbon atoms, and more preferably from 1 to 6 carbon atoms, and from 1 to 4 ring heteroatoms, preferably from 1 to 3 heteroatoms, and more preferably from 1 to 2 heteroatoms selected from the group consisting of nitrogen, sulfur, or oxygen. Cₓ heterocycloalkyl refers to a heterocycloalkyl group having x number of ring atoms including the ring heteroatoms. Heterocycle encompasses single ring or multiple condensed rings, including fused bridged and spiro ring systems. In fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

Examples of heterocyclyl and heteroaryl include, but are not limited to, azetidinyl, pyrrolyl, imidazolyl, pyrazolyl, pyridyl, pyrazyl, pyrimidyl, pyridazyl, indolizyl, isoindolyl, indolyl, dihydroindolyl, indazolyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholinyl, thiomorpholinyl (also referred to as thiamorpholinyl), 1,1-dioxothiomorpholinyl, piperidinyl, pyrrolidinyl, and tetrahydrofuranyl.

“Oxo” refers to the atom (═O) or (O).

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

### FXR Agonists

Suitable FXR agonists that can be used in accordance with the methods described herein include, but are not limited to obeticholic acid, cilofexor, tropifexor, EYP001 (Vonafexor, proposed INN), MET409 (Metacrine), MET642 (Metacrine), EDP-305 (by Enanta), EDP-297 (Enanta), and a compound of formula (I) or a pharmaceutically acceptable salt. The compound of formula (I) is disclosed in US 2010/0152166, the content of which is incorporated by reference in its entirety, and specifically with respect to the compound of formula (I) or a pharmaceutically acceptable salt or enantiomer thereof, as well as methods of making and using the foregoing.

In some embodiments, the FXR agonist is a compound of formula (I)

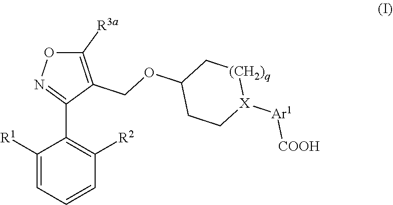

wherein:
q is 1 or 2;
R¹ is chloro, fluoro, or trifluoromethoxy;
R² is hydrogen, chloro, fluoro, or trifluoromethoxy;
R^{3a} is trifluoromethyl, cyclopropyl, or isopropyl;
X is CH or N,
provided that when X is CH, q is 1; and
Ar¹ is indolyl, benzothienyl, naphthyl, phenyl, benzoisothiazolyl, indazolyl, or pyridinyl, each of which is optionally substituted with methyl or phenyl,
or a pharmaceutically acceptable salt thereof.

In some embodiments, the FXR agonist is a compound of formula (I), wherein R¹ is chloro or trifluoromethoxy; and R² is hydrogen or chloro.

In some embodiments, the FXR agonist is a compound of formula (I), wherein R^{3a} is cyclopropyl or isopropyl.

In some embodiments, the FXR agonist is a compound of formula (I), wherein Ar¹ is 5-benzothienyl, 6-benzothienyl, 5-indolyl, 6-indolyl, or 4-phenyl, each of which is optionally substituted with methyl.

In some embodiments, the FXR agonist is a compound of formula (I), wherein q is 1; and X is N.

In some embodiments, the FXR agonist is a compound of formula 1:

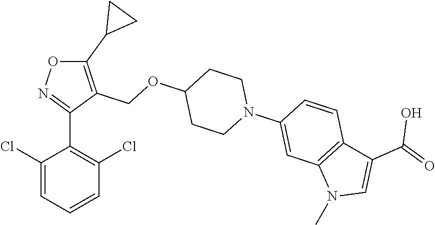 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof. “Compound 1” refers to the compound of formula 1.

### THRβ Agonists

Suitable THRβ agonists that can be used in accordance with the methods described herein include, but are not limited to resmetirom (MGL-3196), VK2809 (by Viking Therapeutics), sobetirome, eprotirome, ALG-055009 (by Aligo), CNPT-101101 (by FronThera Pharmaceuticals), CNPT-101207 (by FronThera Pharmaceuticals), ASC41 (Ascletis), and a compound of formula (II) or a pharmaceutically acceptable salt. The compounds of formula (II) are disclosed in US Application Publication No. 20200190064, the contents of which are incorporated by reference in their entirety, and specifically with respect to the compounds of formula (II), such as compound 2, or a pharmaceutically acceptable salt or enantiomer thereof, as well as methods of making and using the foregoing.

In some embodiments, the THRβ agonist is a compound of Formula (II)

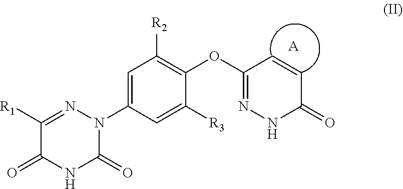

wherein:
R¹ is selected from the group consisting of hydrogen, cyano, substituted or unsubstituted C₁₋₆ alkyl, and substituted or unsubstituted C₃₋₆ cycloalkyl, the substituent being selected from the group consisting of halogen atoms, hydroxy, and C₁₋₆ alkoxy;
R₂ and R₃ are each independently selected from the group consisting of halogen atoms and substituted or unsubstituted C₁₋₆ alkyl, the substituent being selected from the group consisting of halogen atoms, hydroxy, and C₁₋₆ alkoxy;
ring A is a substituted or unsubstituted saturated or unsaturated C₅₋₁₀ aliphatic ring, or a substituted or unsubstituted C₅₋₁₀ aromatic ring, the substituent being one or more substances selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl, and when two substituents are contained, the two substituents can form a ring structure together with the carbon connected thereto; and
the halogen atoms are selected from the group consisting of F, Cl and Br,
or a pharmaceutically acceptable salt thereof.

In some embodiments, the THRβ agonist is a compound of Formula (IIa)

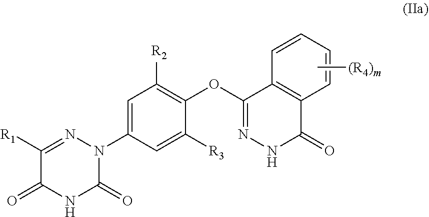

wherein:
R₁ to R₃ are defined as detailed herein for Formula (II);
R₄ is selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl;
m is an integer from the range 1 to 4; and
the halogen atoms are selected from the group consisting of F, Cl and Br.
or a pharmaceutically acceptable salt thereof.

In some embodiments, wherein R₄ is selected from the group consisting of hydrogen, halogen atoms, hydroxy, —OCF₃, C₁₋₆ alkyl, C₁₋₆ alkoxy and C₃₋₆ cycloalkyl; and m is an integer from the range 1 to 3.

In some embodiments, wherein R₁ is selected from the group consisting of hydrogen, cyano, and substituted or unsubstituted C₁₋₆ alkyl, the substituent being selected from the group consisting of halogen atoms, hydroxy, and C₁₋₆ alkoxy; and the halogen atoms are selected from the group consisting of F, Cl and Br.

In some embodiments, the THRβ agonist is a compound of formula 2:

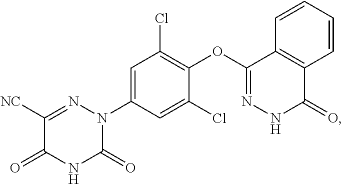 [SMILES: N#Cc1nn(-c2cc(Cl)c(Oc3n[nH]c(=O)c4ccccc34)c(Cl)c2)c(=O)[nH]c1=O]

or a pharmaceutically acceptable salt thereof. “Compound 2” refers to the compound of formula 2.

### Pharmaceutically Acceptable Compositions and Formulations

Pharmaceutically acceptable compositions or simply “pharmaceutical compositions” of any of the compounds detailed herein are embraced by this invention. Thus, the invention includes pharmaceutical compositions comprising an FXR agonist (such as the compound of Formula (I) or a pharmaceutically acceptable salt thereof), a THRβ agonist (such as the compounds of Formula (II) or a pharmaceutically acceptable salt thereof), and a pharmaceutically acceptable carrier or excipient. In some embodiments, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound intends a composition that contains no more than 35% impurity, wherein the impurity denotes a compound other than the compound or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form.

In one variation, the compounds herein are synthetic compounds prepared for administration to an individual such as a human. In another variation, compositions are provided containing a compound in substantially pure form. In another variation, the invention embraces pharmaceutical compositions comprising a compound detailed herein and a pharmaceutically acceptable carrier or excipient. In another variation, methods of administering a compound are provided. The purified forms, pharmaceutical compositions and methods of administering the compounds are suitable for any compound or form thereof detailed herein.

The compounds may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Compounds described herein can be used in the preparation of a formulation, such as a pharmaceutical formulation, by combining the compounds as active ingredients with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Compositions comprising two compounds utilized herein are described. Any of the compounds described herein can be formulated in a tablet in any dosage form described herein.

The present disclosure further encompasses kits (e.g., pharmaceutical packages). The kit provided may comprise the pharmaceutical compositions or the compounds described herein and containers (e.g., drug bottles, ampoules, bottles, syringes and/or subpackages or other suitable containers). In some embodiments, the kit includes a container comprising the FXR agonist (such as the compound of Formula (I) or a pharmaceutically acceptable salt thereof) and the THRβ agonist (such as the compound of (II) or a pharmaceutically acceptable salt thereof). In other embodiments, the kit includes a first container comprising FXR agonist (such as the compound of Formula (I) or a pharmaceutically acceptable salt thereof) and a second container comprising the THRβ agonist (such as the compound of (II) or a pharmaceutically acceptable salt thereof).

In some embodiments, the composition comprises the FXR agonist and the THRβ agonist as described herein. In some embodiments, such a composition includes a compound of formula (I), or a pharmaceutically acceptable salt thereof, and a compound of formula (II), or a pharmaceutically acceptable salt thereof. In some embodiments, provided herein is a dosage form comprises a therapeutically effective amount of a compound of formula (I), or a pharmaceutically acceptable salt thereof, and a therapeutically effective amount of a compound of formula (II), or a pharmaceutically acceptable salt thereof. In some embodiments, the compound of formula (I), or a pharmaceutically acceptable salt thereof, is Compound 1, and the compound of formula (II), or a pharmaceutically acceptable salt thereof, is Compound 2 as described herein.

### Methods of Use and Uses

Compounds and compositions described herein may in some aspects be used in treatment or prevention of liver disorders. In some embodiments, the method of treating or preventing a liver disorder in a patient in need thereof comprises administering to the patient a Farnesoid X Receptor (FXR) agonist and a thyroid hormone receptor beta (THRβ) agonist. In some embodiments, the FXR agonist is a compound of Formula (I), or a pharmaceutically acceptable salt thereof, and the THRβ agonist is a compound of Formula (II), or a pharmaceutically acceptable salt thereof. In one embodiment, the compound of Formula (I), or a pharmaceutically acceptable salt thereof, is Compound 1, and the compound of Formula (II), or a pharmaceutically acceptable salt thereof, is Compound 2 as described herein. Without being bound by theory, it is believed that the combination of an FXR agonist and a THRβ agonist in accordance with the methods described herein may effectively provide treatment as compared to monotherapies and thus reduce dose-dependent adverse effects that may accompany monotherapy treatment.

Liver disorders include, without limitation, liver inflammation, fibrosis, and steatohepatitis. In some embodiments, the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In certain embodiments, the liver disorder is selected from: liver fibrosis, alcohol induced fibrosis, steatosis, alcoholic steatosis, NAFLD, and NASH. In one embodiment, the liver disorder is NASH. In another embodiment, the liver disorder is liver inflammation. In another embodiment, the liver disorder is liver fibrosis. In another embodiment, the liver disorder is alcohol induced fibrosis. In another embodiment, the liver disorder is steatosis. In another embodiment, the liver disorder is alcoholic steatosis. In another embodiment, the liver disorder is NAFLD. In one embodiment, the treatment methods provided herein impedes or slows the progression of NAFLD to NASH. In one embodiment, the treatment methods provided herein impedes or slows the progression of NASH. NASH can progress, e.g., to one or more of liver cirrhosis, hepatic cancer, etc. In some embodiments, the liver disorder is NASH. In some embodiments, the patient has had a liver biopsy. In some embodiments, the method further comprising obtaining the results of a liver biopsy.

In some embodiments, the method of treating a liver disorder in a patient in need thereof, wherein the liver disorder is selected from the group consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

Provided herein are methods of treating or preventing a liver disorder in a patient (e.g., a human patient) in need thereof with an FXR agonist and a THRβ agonist, comprising administering a therapeutically effective amount of the FXR agonist and a therapeutically effective amount of the THRβ agonist, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the FXR agonist is a compound of Formula (I) or a pharmaceutically acceptable salt thereof and the THRβ agonist is a compound of formula (II) or a pharmaceutically acceptable salt thereof. In some embodiments, the compound of formula (I), or a pharmaceutically acceptable salt thereof, is Compound 1, and the compound of formula (II), or a pharmaceutically acceptable salt thereof, is Compound 2 as described herein.

Also provided herein are methods of impeding or slowing the progression of non-alcoholic fatty liver disease (NAFLD) to non-alcoholic steatohepatitis (NASH) in a patient (e.g., a human patient) in need thereof comprising administering an FXR agonist (such as the compound of Formula (I) or a pharmaceutically acceptable salt thereof) and a THRβ agonist (such as the compounds of Formula (II) or a pharmaceutically acceptable salt thereof). In some embodiments, the methods comprises administering a therapeutically effective amount of a compound of formula (I), or a pharmaceutically acceptable salt thereof, and a therapeutically effective amount of a compound of formula (II) or a pharmaceutically acceptable salt thereof. Also provided herein are methods of impeding or slowing the progression of NASH in a patient (e.g., a human patient) in need thereof comprising administering an FXR agonist (such as the compound of Formula (I) or a pharmaceutically acceptable salt thereof) and a THRβ agonist (such as the compounds of Formula (II) or a pharmaceutically acceptable salt thereof). In some embodiments, the methods comprises administering a therapeutically effective amount of a compound of formula (I), or a pharmaceutically acceptable salt thereof, and a therapeutically effective amount of a compound of formula (II) or a pharmaceutically acceptable salt thereof.

Further, pruritus is a well-documented adverse effect of several FXR agonists and can result in patient discomfort, a decrease in patient quality of life, and an increased likelihood of ceasing treatment. Pruritus is particularly burdensome for indications, such as those described herein, including NASH, for which chronic drug administration is likely. The tissue specificity of the compound of formula (I), in particular the preference for liver over skin tissue is a striking and unpredicted observation that makes it more likely that the compound will not cause pruritus in the skin, a theory that has been substantiated by human trials thus far.

Accordingly, provided herein are methods of treating a liver disorder in a patient in need thereof (e.g., a human patient) with an FXR agonist and a THRβ agonist, wherein the FXR is a compound of Formula (I), or a pharmaceutically acceptable salt thereof, which preferentially distributes in liver tissue over one or more of kidney, lung, heart, and skin.

In some embodiments, the administration results in a liver concentration to plasma concentration ratio of the compound of Formula (I) of 10 or greater, such as 11 or greater, 12 or greater, 13 or greater, 14 or greater, or 15 or greater.

In some embodiments, the administration does not result in pruritus in the patient greater than Grade 2 in severity. In some embodiments, the administration does not result in pruritus in the patient greater than Grade 1 in severity. In some embodiments, the administration does not result in pruritus in the patient. The grading of adverse effects is known. According to Version 5 of the Common Terminology Criteria for Adverse Events (published Nov. 27, 2017), Grade 1 pruritus is characterized as “Mild or localized; topical intervention indicated.” Grade 2 pruritus is characterized as “Widespread and intermittent; skin changes from scratching (e.g., edema, papulation, excoriations, lichenification, oozing/crusts); oral intervention indicated; limiting instrumental ADL.” Grade 3 pruritus is characterized as “Widespread and constant; limiting self care ADL or sleep; systemic corticosteroid or immunosuppressive therapy indicated.” Activities of daily living (ADL) are divided into two categories: “Instrumental ADL refer to preparing meals, shopping for groceries or clothes, using the telephone, managing money, etc.,” and “Self care ADL refer to bathing, dressing and undressing, feeding self, using the toilet, taking medications, and not bedridden.” Accordingly, provided herein are methods of treating a liver disorder in a patient (e.g., a human patient) in need thereof with an FXR agonist that does not result in detectable pruritus in the patient in need thereof.

In some embodiments, provided herein are methods of treating a liver disorder in a patient in need thereof with an FXR agonist (such as the compound of Formula (I) or a pharmaceutically acceptable salt thereof) and a THRβ agonist (such as the compounds of Formula (II) or a pharmaceutically acceptable salt thereof), wherein the FXR agonist does not activate TGR5 signaling. In some embodiments, the level of an FXR-regulated gene is increased. In some embodiments, the level of small heterodimer partner (SHP), bile salt export pump (BSEP) and fibroblast growth factor 19 (FGF19) is increased.

In some embodiments, provided herein a method of reducing liver damage comprising administering an FXR agonist (such as the compound of Formula (I) or a pharmaceutically acceptable salt thereof) and a THRβ agonist (such as the compounds of Formula (II) or a pharmaceutically acceptable salt thereof), to an individual in need thereof, wherein fibrosis is reduced. In some embodiments, the level of expression of one or more markers for fibrosis is reduced. In some embodiments, the level of Ccr2, Col1a1, Col1a2, Col1a3, Cxcr3, Dcn, Hgf, Il1a, Inhbe, Lox, Loxl1, Loxl2, Loxl3, Mmp2, Pdgfb, Plau, Serpine1, Perpinh1, Snai, Tgfb1, Tgfb3, Thbs1, Thbs2, Timp2, and/or Timp3 expression is reduced. In some embodiments the level of collagen is reduced. In some embodiments, the level of collagen fragments is reduced. In some embodiments, the level of expression of the fibrosis marker is reduced at least 2, at least 3, at least 4, or at least 5-fold. In some embodiments, the level of expression of the fibrosis marker is reduced about 2-fold, about 3-fold, about 4-fold, or about 5-fold.

In some embodiments, provided herein a method of reducing liver damage comprising administering an FXR agonist (such as the compound of Formula (I) or a pharmaceutically acceptable salt thereof) and a THRβ agonist (such as the compounds of Formula (II) or a pharmaceutically acceptable salt thereof), to an individual in need thereof, wherein inflammation is reduced. In some embodiments, one or more markers of inflammation are reduced. In some embodiments, the level of expression of Adgre1, Ccr2, Ccr5, Il1A, and/or Tlr4 is reduced. In some embodiments, the level of expression of the inflammation marker is reduced at least 2-, at least 3-, at least 4-, or at least 5-fold. In some embodiments, the level of expression of the fibrosis marker is reduced about 2-fold, about 3-fold, about 4-fold, or about 5-fold.

In a patient, alkaline phosphatase, gamma-glutamyl transferase (GGT), alanine aminotransferase (ALT) and/or aspartate aminotransferase (AST) levels can be elevated. In some embodiments, provided herein a method of reducing liver damage comprising administering an FXR agonist (such as the compound of Formula (I) or a pharmaceutically acceptable salt thereof) and a THRβ agonist (such as the compounds of Formula (II) or a pharmaceutically acceptable salt thereof), wherein the GGT, ALT, and/or AST levels are elevated prior to treatment with the FXR agonist. In some embodiments, the FXR agonist is a compound of Formula (I) or a pharmaceutically acceptable salt thereof. In some embodiments, the patient's ALT level is about 2-4-fold greater than the upper limit of normal levels. In some embodiments, the patient's AST level is about 2-4-fold greater than the upper limit of normal levels. In some embodiments, the patient's GGT level is about 1.5-3-fold greater than the upper limit of normal levels. In some embodiments, the patient's alkaline phosphatase level is about 1.5-3-fold greater than the upper limit of normal levels. Methods of determining the levels of these molecules are well known. Normal levels of ALT in the blood range from about 7-56 units/liter. Normal levels of AST in the blood range from about 10-40 units/liter. Normal levels of GGT in the blood range from about 9-48 units/liter. Normal levels of alkaline phosphatase in the blood range from about 53-128 units/liter for a 20- to 50-year-old man and about 42-98 units/liter for a 20- to 50-year-old woman.

Accordingly, in some embodiments, a compound of Formula (I), or a pharmaceutically acceptable salt thereof, reduces level of AST, ALT, and/or GGT in an individual having elevated AST, ALT, and/or GGT levels. In some embodiments, the level of ALT is reduced at least 2-, at least 3-, at least 4-, or at least 5-fold. In some embodiments, the level of ALT is reduced about 2- to about 5-fold. In some embodiments, the level of AST is reduced at least 2-, at least 3-, at least 4-, or at least 5-fold. In some embodiments, the level of AST is reduced about 1.5 to about 3-fold. In some embodiments, the level of GGT is reduced at least 2, at least 3, at least 4, or at least 5-fold. In some embodiments, the level of GGT is reduced about 1.5 to about 3-fold.

In some embodiments, the patient is a human. Obesity is highly correlated with NAFLD and NASH, but lean people can also be affected by NAFLD and NASH. Accordingly, in some embodiments, the patient is obese. In some embodiments, the patient is not obese. Obesity can be correlated with or cause other diseases as well, such as diabetes mellitus or cardiovascular disorders. Accordingly, in some embodiments, the patient also has diabetes mellitus and/or a cardiovascular disorder. Without being bound by theory, it is believed that comorbidities, such as obesity, diabetes mellitus, and cardiovascular disorders can make NAFLD and NASH more difficult to treat. Conversely, the only currently recognized method for addressing NAFLD and NASH is weight loss, which would likely have little to no effect on a lean patient.

The risk for NAFLD and NASH increases with age, but children can also suffer from NAFLD and NASH, with literature reporting of children as young as 2 years old (Schwimmer, et al., Pediatrics, 2006, 118:1388-1393). In some embodiments, the patient is 2-17 years old, such as 2-10, 2-6, 2-4, 4-15, 4-8, 6-15, 6-10, 8-17, 8-15, 8-12, 10-17, or 13-17 years old. In some embodiments, the patient is 18-64 years old, such as 18-55, 18-40, 18-30, 18-26, 18-21, 21-64, 21-55, 21-40, 21-30, 21-26, 26-64, 26-55, 26-40, 26-30, 30-64, 30-55, 30-40, 40-64, 40-55, or 55-64 years old. In some embodiments, the patient is 65 or more years old, such as 70 or more, 80 or more, or 90 or more.

NAFLD and NASH are common causes of liver transplantation, but patients that already received one liver transplant often develop NAFLD and/or NASH again. Accordingly, in some embodiments, the patient has had a liver transplant.

In some embodiments, treatment in accordance with the methods provided herein results in a reduced NAFLD Activity (NAS) score in a patient. For example, in some embodiments, steatosis, inflammation, and/or ballooning is reduced upon treatment. In some embodiments, the methods of treatment provided herein reduce liver fibrosis. In some embodiments, the methods reduce serum triglycerides. In some embodiments, the methods reduce liver triglycerides.

In some embodiments, the patient is at risk of developing an adverse effect prior to the administration in accordance with the methods provided herein. In some embodiments, the adverse effect is an adverse effect which affects the kidney, lung, heart, and/or skin. In some embodiments, the adverse effect is pruritus.

In some embodiments, the patient has had one or more prior therapies. In some embodiments, the liver disorder progressed during the therapy. In some embodiments, the patient suffered from pruritus during at least one of the one or more prior therapies.

In some embodiments, the methods described herein do not comprise treating pruritus in the patient. In some embodiments, the methods do not comprise administering an antihistamine, an immunosuppressant, a steroid (such as a corticosteroid), rifampicin, an opioid antagonist, or a selective serotonin reuptake inhibitor (SSRI).

In some embodiments, the therapeutically effective amounts of either the FXR agonist or the THRβ agonist, or both are below the level that induces an adverse effect in the patient, such as below the level that induces pruritus, such as grade 2 or grade 3 pruritus.

In some embodiments, the FXR agonist and the THRβ agonist are administered simultaneously. In some such embodiments, the FXR agonist and the THRβ agonist can be provided in a single pharmaceutical composition. In other embodiments, the FXR agonist and the THRβ agonist are administered sequentially.

Also provided herein are dosing regimens for administering an FXR agonist (such as the compound of Formula (I) or a pharmaceutically acceptable salt thereof) and a THRβ agonist (such as the compounds of Formula (II) or a pharmaceutically acceptable salt thereof), to an individual in need thereof. In some embodiments, the therapeutically effective amounts of the FXR agonist (such as the compound of Formula (I) or a pharmaceutically acceptable salt thereof) and the THRβ agonist (such as the compounds of Formula (II) or a pharmaceutically acceptable salt thereof) are independently 500 μg/day-600 mg/day. In some embodiments, the therapeutically effective amounts are independently 500 μg/day-300 mg/day. In some embodiments, the therapeutically effective amounts are independently 500 μg/day-150 mg/day. In some embodiments, the therapeutically effective amounts are independently 500 μg/day-100 mg/day. In some embodiments, the therapeutically effective amounts are independently 500 μg/day-20 mg/day. In some embodiments, the therapeutically effective amounts are independently 1 mg/day-600 mg/day. In some embodiments, the therapeutically effective amounts are independently 1 mg/day-300 mg/day. In some embodiments, the therapeutically effective amounts are independently 1 mg/day-150 mg/day. In some embodiments, the therapeutically effective amounts are independently 1 mg/day-100 mg/day. In some embodiments, the therapeutically effective amounts are independently 1 mg/day-20 mg/day. In some embodiments, the therapeutically effective amounts are independently 5 mg/day-300 mg/day. In some embodiments, the therapeutically effective amounts are independently 5 mg/day-150 mg/day. In some embodiments, the therapeutically effective amounts are independently 5 mg/day-100 mg/day. In some embodiments, the therapeutically effective amounts are independently 5 mg/day-20 mg/day. In some embodiments, the therapeutically effective amounts are independently 5 mg/day-15 mg/day. In some embodiments, the therapeutically effective amounts are independently 10 mg/day-300 mg/day. In some embodiments, the therapeutically effective amounts are independently 10 mg/day-150 mg/day. In some embodiments, the therapeutically effective amounts are independently 10 mg/day-100 mg/day. In some embodiments, the therapeutically effective amounts are independently 10 mg/day-30 mg/day. In some embodiments, the therapeutically effective amounts are independently 10 mg/day-20 mg/day. In some embodiments, the therapeutically effective amounts are independently 10 mg/day-15 mg/day. In some embodiments, the therapeutically effective amounts are independently 25 mg/day-300 mg/day. In some embodiments, the therapeutically effective amounts are independently 25 mg/day-150 mg/day. In some embodiments, the therapeutically effective amounts are independently 25 mg/day-100 mg/day. In some embodiments, the therapeutically effective amounts are independently 500 μg/day-5 mg/day. In some embodiments, the therapeutically effective amounts are independently 500 μg/day-4 mg/day. In some embodiments, the therapeutically effective amounts are independently 5 mg/day-600 mg/day. In another embodiment, the therapeutically effective amounts are independently 75 mg/day-600 mg/day. In one embodiment, the compound of Formula (I), or a pharmaceutically acceptable salt thereof, is Compound 1, and the compound of Formula (II), or a pharmaceutically acceptable salt thereof, is Compound 2 as described herein.

The dosage amount of a compound as described herein is determined based on the free base of a compound. In some embodiments, about 1 mg to about 30 mg of the FXR agonist (such as the compound of Formula (I) or a pharmaceutically acceptable salt thereof) is administered to the individual. In some embodiments, about 1 mg to about 5 mg of the compound is administered to the individual. In some embodiments, about 1 mg to about 3 mg of the compound is administered to the individual. In some embodiments, about 5 mg to about 10 mg of the compound is administered to the individual. In some embodiments, about 10 mg to about 15 mg of the compound is administered to the individual. In some embodiments, about 15 mg to about 20 mg of the compound is administered to the individual. In some embodiments, about 20 mg to about 25 mg of the compound is administered to the individual. In some embodiments, about 25 mg to about 30 mg of the compound is administered to the individual. In some embodiments, about 1 mg of the compound is administered to the individual. In some embodiments, about 2 mg of the compound is administered to the individual. In some embodiments, about 3 mg of the compound is administered to the individual. In some embodiments, about 4 mg of the compound is administered to the individual. In some embodiments, about 5 mg of the compound is administered to the individual. In some embodiments, about 6 mg of the compound is administered to the individual. In some embodiments, about 7 mg of the compound is administered to the individual. In some embodiments, about 8 mg of the compound is administered to the individual. In some embodiments, about 9 mg of the compound is administered to the individual. In some embodiments, about 10 mg of the compound is administered to the individual. In some embodiments, about 15 mg of the compound is administered to the individual. In some embodiments, about 20 mg of the compound is administered to the individual. In some embodiments, about 25 mg of the compound is administered to the individual. In some embodiments, about 30 mg of the compound is administered to the individual. In one embodiment, the compound is Compound 1 as described herein.

In some embodiments, about 0.5 mg to about 100 mg of the THRβ agonist (such as the compound of Formula (II) or a pharmaceutically acceptable salt thereof) is administered to the individual. In some embodiments, about 1 mg to about 5 mg of the compound is administered to the individual. In some embodiments about 1 mg to about 30 mg of the compound is administered to the individual. In some embodiments about 1 mg to about 3 mg of the compound is administered to the individual. In some embodiments about 5 mg to about 10 mg of the compound is administered to the individual. In some embodiments, about 10 mg to about 15 mg of the compound is administered to the individual. In some embodiments, about 15 mg to about 20 mg of the compound is administered to the individual. In some embodiments, about 20 mg to about 25 mg of the compound is administered to the individual. In some embodiments, about 25 mg to about 30 mg of the compound is administered to the individual. In some embodiments, about 1 mg of the compound is administered to the individual. In some embodiments, about 2 mg of the compound is administered to the individual. In some embodiments, about 3 mg of the compound is administered to the individual. In some embodiments, about 4 mg of the compound is administered to the individual. In some embodiments, about 5 mg of the compound is administered to the individual. In some embodiments, about 6 mg of the compound is administered to the individual. In some embodiments, about 7 mg of the compound is administered to the individual. In some embodiments, about 8 mg of the compound is administered to the individual. In some embodiments, about 9 mg of the compound is administered to the individual. In some embodiments, about 10 mg of the compound is administered to the individual. In some embodiments, about 15 mg of the compound is administered to the individual. In some embodiments, about 20 mg of the compound is administered to the individual. In some embodiments, about 25 mg of the compound is administered to the individual. In some embodiments, about 30 mg of the compound is administered to the individual. In one embodiment, the compound is Compound 2 as described herein.

The treatment period generally can be one or more weeks. In some embodiments, the treatment period is at least 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is from about a week to about a month, from about a month to about a year, from about a year to about several years. In some embodiments, the treatment period at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is the remaining lifespan of the patient.

The administration of the FXR agonist (such as the compound of Formula (I) or a pharmaceutically acceptable salt thereof) and the THRβ agonist (such as the compound of (II) or a pharmaceutically acceptable salt thereof) can independently be once daily, twice daily or every other day, for a treatment period of one or more weeks. In some embodiments, the administration comprises administering both compounds daily for a treatment period of one or more weeks. In some embodiments, the administration comprises administering both compounds twice daily for a treatment period of one or more weeks. In some embodiments, the administration comprises administering both compounds every other day for a treatment period of one or more weeks.

In some embodiments, the FXR agonist (such as the compound of Formula (I) or a pharmaceutically acceptable salt thereof) and the THRβ agonist (such as the compound of (II) or a pharmaceutically acceptable salt thereof) are administered to the individual once per day for at least seven days, wherein the daily amounts are independently in a range of about 1 mg to about 10 mg, about 1 mg to about 5 mg or about 1 mg to about 3 mg, or about any one of 1, 2, 3, 4, 5, 6, 7, 8, 9, or 10 mg. In some embodiments, both compounds are administered to the individual once per day for at least 14 days, wherein the daily amounts are independently in a range of about 1 mg to about 10 mg, about 1 mg to about 5 mg or about 1 mg to about 3 mg or about any one of 1, 2, 3, 4, 5, 6, 7, 8, 9, or 10 mg. In some embodiments, both compounds are administered to the individual once per day for a period of between one and four weeks, wherein the daily amounts are independently in a range of about 1 mg to about 10 mg, about 1 mg to about 5 mg or about 1 mg to about 3 mg or about any one of 1, 2, 3, 4, 5, 6, 7, 8, 9, or 10 mg.

When administered in combination with a THRβ agonist, the FXR agonist and/or the THRβ agonist can be administered at doses that are typically administered when either agent is administered alone. Alternatively, as a result of the synergy observed with the combination, the FXR agonist and/or the THRβ agonist can be administered at doses that are lower than doses when either agent is administered alone. For instance, in embodiments wherein the FXR agonist is a compound of Formula (I) (e.g., Compound 1) or a pharmaceutically acceptable salt thereof, a therapeutic dose of the compound of Formula (I) to a human patient is typically from about 5 mg to about 15 mg daily administered orally. Hence, in particular embodiments, when administered in combination with a THRβ agonist, the compound of Formula (I) or a pharmaceutically acceptable salt thereof can be administered at an oral dose of from about 5 mg to about 15 mg (e.g., 5 mg, 6 mg, 7 mg, 8 mg, 9 mg, 10 mg, 11 mg, 12 mg, 13 mg, 14 mg or 15 mg) or can be administered at a lower dose. For instance, when administered in combination with a THRβ agonist, the compound of Formula (I) or a pharmaceutically acceptable salt thereof can be administered orally at a dose of from about 1 mg to about 15 mg daily, from about 1 mg to about 4.9 mg daily, from about 1 mg to about 4 mg daily, from about 2 mg to about 4 mg daily, or of any of 1, 1.5, 2, 2.5, 3, 3.5, 4, 4.5, 4.9, 5, 6, 7, 8, 9, 10, 11, 12, 13, 14, or 15 mg daily.

In embodiments wherein the THRβ agonist is a compound of formula (II) (e.g., Compound 2) or a pharmaceutically acceptable salt thereof, a therapeutic dose of the compound to a human patient is typically from about 3 mg to about 90 mg daily administered orally. In particular embodiments, when administered in combination with a FXR agonist, the compound of formula (II) or a pharmaceutically acceptable salt thereof can be administered at an oral dose of from about 3 mg to about 90 mg (e.g., 3 mg, 5 mg, 10 mg, 20 mg, 30 mg, 40 mg, 50 mg, 60 mg, 70 mg, 80 mg or 90 mg) or can be administered at a lower dose. For instance, when administered in combination with a FXR agonist, the compound of formula (II) or a pharmaceutically acceptable salt thereof can be administered orally at a dose of from about 0.5 mg to about 30 mg daily, from about 0.5 mg to about 25 mg daily, from about 0.5 mg to about 20 mg daily, from about 0.5 mg to about 15 mg daily, from about 0.5 mg to about 10 mg daily, from about 0.5 mg to about 5 mg daily, from about 0.5 mg to about 3 mg daily, or from about 1 mg to about 3 mg daily.

In particular embodiments wherein the FXR agonist is a compound of formula (I) (e.g., Compound 1) or a pharmaceutically acceptable salt thereof and the THRβ agonist is a compound of formula (II) (e.g., Compound 2) or a pharmaceutically acceptable salt thereof, the dose of each individual compound can be administered as set forth above. For instance, in some embodiments, the compound of formula (I) or a pharmaceutically acceptable salt thereof, is administered at a dose from about 1 mg to about 15 mg daily in combination with the compound of formula (II) or a pharmaceutically acceptable salt thereof administered at a dose of from about 0.5 mg to about 90 mg daily. In some embodiments, the compound of formula (I) or a pharmaceutically acceptable salt thereof is administered at a dose from about 5 mg to about 15 mg daily in combination with the compound of formula (II) or a pharmaceutically acceptable salt thereof administered at a dose of from about 0.5 mg to about 10 mg daily, from about 10 mg to about 20 mg daily, from about 10 mg to about 40 mg daily, from about 20 mg to about 50 mg daily or from about 50 mg to about 90 mg daily. In some embodiments, the compound of formula (I) or a pharmaceutically acceptable salt thereof is administered at a dose from about 1 mg to about 5 mg daily in combination with the compound of formula (II) or a pharmaceutically acceptable salt thereof administered at a dose of from about 0.5 mg to about 10 mg daily, from about 10 mg to about 20 mg daily, from about 10 mg to about 40 mg daily, from about 20 mg to about 50 mg daily or from about 50 mg to about 90 mg daily.

In some embodiments, the amount of the FXR agonist (such as the compound of Formula (I) or a pharmaceutically acceptable salt thereof) and the amount of the THRβ agonist (such as the compound of (II) or a pharmaceutically acceptable salt thereof) administered on day 1 of the treatment period are greater than or equal to the amounts administered on all subsequent days of the treatment period. In some embodiments, the amounts administered on day 1 of the treatment period are equal to the amounts administered on all subsequent days of the treatment period.

In some embodiments, the administration modulates one or more of the following: a metabolic pathway, bile secretion, retinol metabolism, drug metabolism-cytochrome P450, fat digestion and absorption, glycerolipid metabolism, chemical carcinogenesis, glyceropholipid metabolism, nicotine addiction, linoleic acid metabolism, ABC transporters, metabolism of xenobiotics by cytochrome P450, sphingolipid metabolism, glutathione metabolism, folate biosynthesis, morphine addiction, glycosphingolipid biosynthesis-lacto and neolacto series, arachidonic acid metabolism, tyrosine metabolism, maturity onset diabetes of the young, DNA replication, cholesterol metabolism, drug metabolism-other enzymes, and ether lipid metabolism. In some embodiments, the administration modulates one or more of the following: a metabolic pathway, retinol metabolism, fat digestion and absorption, glycerolipid metabolism, chemical carcinogenesis, glyceropholipid metabolism, ABC transporters, metabolism of xenobiotics by cytochrome P450, sphingolipid metabolism, glutathione metabolism, folate biosynthesis, and morphine addiction. In some embodiments, the administration modulates expression of one or more of the following: Abcb4, Apoa5, Cyp7a1, Cyp8b1, Nr0b2, and Sic51b.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) enriches GO terms associated with immune-related biological processes. Methods of assessing GO term enrichment are known to the skilled artisan and may include detection of (a) increased expression of a set of functionally related genes, or (b) reduced expression of a set of functionally related genes. For instance, reduced expression of genes associated with immune pathways results in significant enrichment of immune-related GO terms, as described in Examples 13-15. In some embodiments, administration with the combination enriches immune-related biological processes as compared to administration with a monotherapy of the FXR agonist or the THRβ agonist. In some embodiments, administration with the combination enriches a larger number of immune-related biological processes ≥1.5-fold as compared to administration with a monotherapy of the FXR agonist or the THRβ agonist. In some embodiments, administration with the combination reduces inflammation in the individual. In some embodiments, administration with the combination reduces inflammation in the individual as compared to administration with a monotherapy of the FXR agonist or the THRβ agonist. In some embodiments, administration with the combination provides synergistic reduction in inflammation in the individual as compared to administration with a monotherapy of the FXR agonist or the THRβ agonist. Thus it is to be understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises enriching one or more immune-related biological processes, reducing gene expression of one or more immune-related genes, and/or reducing inflammation. In some embodiments, the one or more immune-related biological processes are selected from the following GO term IDs: GO:0006955, GO:0006954, GO:0002274, GO:0002376, GO:0045321, GO:0002684, GO:0050900, GO:0050776, GO:0002682, GO:0002269, GO:0097529, GO:0030595, GO:0050778, GO:0045087, GO:0007159, GO:0070661, GO:0150076, GO:0002685, GO:0002443, GO:0002263, GO:0002366, GO:0002694, GO:0050727, GO:0002696, GO:0002250, GO:0002687, GO:0002252, GO:0050729, GO:0002757, GO:0070663, GO:0002764, GO:0070486, GO:0002703, GO:0002699, GO:1903039, GO:1903037, GO:0002275, GO:0002690, GO:0002521, GO:0002253, GO:0002444, GO:0002705, GO:0002526, GO:0043299, GO:0002688, GO:0002429, GO:0002886, GO:0002768, and GO:0070665. In some embodiments, the one or more immune-related biological processes are selected from the following GO term IDs: GO:0006955, GO:0006954, GO:0002274, GO:0002376, GO:0045321, GO:0002684, GO:0050900, GO:0050776, GO:0002682, GO:0002269, GO:0097529, GO:0030595, GO:0050778, GO:0045087, GO:0007159, GO:0070661.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) enriches GO-terms associated with leukocyte-associated biological processes. Methods of assessing GO term enrichment are known to the skilled artisan and may include detection of (a) increased expression of a set of functionally related genes, or (b) reduced expression of a set of functionally related genes. For instance, reduced expression of genes associated with leukocyte-associated biological processes results in significant enrichment of leukocyte-associated GO terms, as described in Examples 13-15. In some embodiments, administration with the combination enriches leukocyte-associated biological processes as compared to administration with a monotherapy of the FXR agonist or the THRβ agonist. In some embodiments, administration with the combination enriches leukocyte-associated biological processes ≥1.5-fold as compared to administration with a monotherapy of the FXR agonist or the THRβ agonist. In some embodiments, administration with the combination reduces leukocyte activation in the individual. In some embodiments, administration with the combination reduces leukocyte activation in the individual as compared to administration with a monotherapy of the FXR agonist or the THRβ agonist. In some embodiments, administration with the combination decreases leukocyte count in the individual as compared to administration with a monotherapy of the FXR agonist or the THRβ agonist. In some embodiments, administration with the combination provides synergistic reduction of leukocyte activation in the individual as compared to administration with a monotherapy of the FXR agonist or the THRβ agonist. Thus it is to be understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises enriching one or more leukocyte-associated biological processes, reducing gene expression of one or more leukocyte-associated genes, decreasing leukocyte count, or reducing leukocyte function.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) enriches GO-terms associated with both immune-related biological processes and leukocyte-associated biological processes. In some embodiments, administration with the combination enriches immune-related biological processes and leukocyte-associated biological processes as compared to administration with a monotherapy of the FXR agonist or the THRβ agonist. In some embodiments, administration with the combination enriches immune-related biological processes and leukocyte-associated biological processes ≥1.5-fold as compared to administration with a monotherapy of the FXR agonist or the THRβ agonist. In some embodiments, administration with the combination reduces inflammation or leukocyte activation or decreases leukocyte recruitment in the liver in the individual as compared to administration with a monotherapy of the FXR agonist or the THRβ agonist. In some embodiments, administration with the combination reduces inflammation and leukocyte activation in the individual as compared to administration with a monotherapy of the FXR agonist or the THRβ agonist. In some embodiments, administration with the combination reduces inflammation and decreases leukocyte recruitment to the liver in the individual. In some embodiments, administration with the combination provides synergistic reduction of inflammation or leukocyte function or decreases leukocyte count in the individual as compared to administration with a monotherapy of the FXR agonist or the THRβ agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises: (1) enriching one or more immune-related biological processes, decreasing gene expression of one or more immune-related genes, or reducing inflammation; and (2) enriching one or more leukocyte-associated biological processes, reducing gene expression of one or more leukocyte-associated genes, decreasing leukocyte recruitment to the liver, or reducing leukocyte function.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) results in differential expression of genes. In some embodiments, administration with the combination results in differential expression of genes as compared to administration with a monotherapy of the FXR agonist or the THRβ agonist. In some embodiments, administration with the combination results in differential expression of immune-related genes. In some embodiments, administration with the combination results in differential expression of immune-related genes as compared to administration with a monotherapy of the FXR agonist or the THRβ agonist. In some embodiments, administration with the combination results in differential expression of immune-related genes ≥1.5-fold as compared to administration with a monotherapy of the FXR agonist or the THRβ agonist. In some embodiments, administration with the combination results in differential expression of leukocyte-associated genes. In some embodiments, administration with the combination results in differential expression of leukocyte-associated genes as compared to administration with a monotherapy of the FXR agonist or the THRβ agonist. In some embodiments, administration with the combination results in differential expression of leukocyte-associated genes ≥1.5-fold as compared to administration with a monotherapy of the FXR agonist or the THRβ agonist. In some embodiments, administration with the combination provides a synergistic increase in the number of differentially expressed genes in the individual as compared to administration with a monotherapy of the FXR agonist or the THRβ agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing gene expression of one or more immune-related genes and/or one or more leukocyte-associated genes.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) decreases steatosis in the individual. Methods of assessing steatosis are known to the skilled artisan and may include histological analysis and assignment of histological score. In some embodiments, administration with the combination decreases steatosis in the individual as compared to administration with a monotherapy of the FXR agonist or the THRβ agonist. In some embodiments, administration with the combination decreases steatosis in the individual comparably as well as administration with a monotherapy of the FXR agonist or the THRβ agonist. In some embodiments, administration with the combination provides a synergistic decrease in steatosis in the individual as compared to administration with a monotherapy of the FXR agonist or the THRβ agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing histological markers associated with steatosis.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) decreases liver inflammation in the individual. Methods of assessing liver inflammation are known to the skilled artisan and may include histological analysis and assignment of histological score of lobular inflammation. In some embodiments, administration with the combination decreases liver inflammation in the individual as compared to administration with a monotherapy of the FXR agonist or the THRβ agonist. In some embodiments, administration with the combination decreases liver inflammation in the individual comparably as well as administration with a monotherapy of the FXR agonist or the THRβ agonist. In some embodiments, administration with the combination provides a synergistic decrease in liver inflammation in the individual as compared to administration with a monotherapy of the FXR agonist or the THRβ agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing lobular inflammation or histological markers associated with lobular inflammation.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) decreases liver fibrosis in the individual. Methods of assessing liver fibrosis are known to the skilled artisan and may include histological analysis. In some embodiments, administration with the combination decreases liver fibrosis in the individual as compared to administration with a monotherapy of the FXR agonist or the THRβ agonist. In some embodiments, administration with the combination decreases liver fibrosis in the individual comparably as well as administration with a monotherapy of the FXR agonist or the THRβ agonist. In some embodiments, administration with the combination provides a synergistic decrease in liver fibrosis in the individual as compared to administration with a monotherapy of the FXR agonist or the THRβ agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing fibrosis or histological markers associated with fibrosis.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) decreases at least one or at least two of liver steatosis, inflammation, and fibrosis in the individual. In some embodiments, administration with the combination decreases at least one or at least two of liver steatosis, inflammation, and fibrosis in the individual as compared to administration with a monotherapy of the FXR agonist or the THRβ agonist. In some embodiments, administration with the combination decreases liver steatosis, inflammation, and fibrosis in the individual. In some embodiments, administration with the combination decreases liver steatosis, inflammation, and fibrosis in the individual as compared to administration with a monotherapy of the FXR agonist or the THRβ agonist. In some embodiments, administration with the combination provides a synergistic decrease in at least one or at least two of steatosis, inflammation, and fibrosis in the individual as compared to administration with a monotherapy of the FXR agonist or the THRβ agonist. In some embodiments, administration with the combination provides a synergistic decrease in steatosis, inflammation, and fibrosis in the individual as compared to administration with a monotherapy of the FXR agonist or the THRβ agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing at least one or at least two of steatosis, lobular inflammation, fibrosis, or histological markers of any of the foregoing.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) decreases serum triglycerides in the individual. In some embodiments, administration with the combination decreases serum triglycerides in the individual as compared to administration with a monotherapy of the FXR agonist or the THRβ agonist. In some embodiments, administration with the combination decreases serum triglycerides in the individual comparably as well as administration with a monotherapy of the FXR agonist or the THRβ agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing serum triglycerides.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) decreases serum total cholesterol in the individual. In some embodiments, administration with the combination decreases serum total cholesterol in the individual as compared to administration with a monotherapy of the FXR agonist or the THRβ agonist. In some embodiments, administration with the combination decreases serum total cholesterol in the individual comparably as well as administration with a monotherapy of the FXR agonist or the THRβ agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing serum cholesterol.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) decreases serum alanine aminotransferase in the individual. In some embodiments, administration with the combination decreases serum alanine aminotransferase in the individual as compared to administration with a monotherapy of the FXR agonist or the THRβ agonist. In some embodiments, administration with the combination decreases serum alanine aminotransferase in the individual comparably as well as administration with a monotherapy of the FXR agonist or the THRβ agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing serum alanine aminotransferase.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) decreases at least one or at least two of serum triglycerides, total cholesterol, and alanine aminotransferase in the individual. In some embodiments, administration with the combination decreases at least one or at least two of serum triglycerides, total cholesterol, and alanine aminotransferase in the individual as compared to administration with a monotherapy of the FXR agonist or the THRβ agonist. In some embodiments, administration with the combination decreases serum triglycerides, total cholesterol, and alanine aminotransferase in the individual. In some embodiments, administration with the combination decreases serum triglycerides, total cholesterol, and alanine aminotransferase in the individual as compared to administration with a monotherapy of the FXR agonist or the THRβ agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing at least one or at least two of serum triglycerides, total cholesterol, and alanine aminotransferase.

In some embodiments, administration with the combination of the FXR agonist (such as the compound of Formula (I) or a pharmaceutically acceptable salt thereof) and the THRβ agonist (such as the compound of (II) or a pharmaceutically acceptable salt thereof) decreases expression of one or more fibrosis- and/or inflammation-associated genes in the individual. Genes associated with fibrosis and/or inflammation include, but are not limited to, Col1a1, Col3a1, Mmp2, Lgals3, Cd68, and Ccr2. Methods of assessing expression are known to the skilled artisan and may include RNAseq. In some embodiments, administration with the combination decreases expression of at least 1, at least 2, at least 3, at least 4, at least 5, or at least 6 genes associated with fibrosis and/or inflammation. In some embodiments, administration with the combination decreases expression of at least 1, at least 2, at least 3, at least 4, or at least 5 genes selected from Col1a1, Col3a1, Mmp2, Lgals3, Cd68, and Ccr2. In some embodiments, administration with the combination decreases expression of Col1a1, Col3a1, Mmp2, Lgals3, Cd68, and Ccr2. In some embodiments, administration with the combination decreases expression of fibrosis- and/or inflammation-associated genes in the individual as compared to administration with a monotherapy of the FXR agonist or the THRβ agonist. In some embodiments, administration with the combination decreases expression of fibrosis- and/or inflammation-associated genes in the individual comparably as well as administration with a monotherapy of the FXR agonist or the THRβ agonist. Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises decreasing expression of at least 1, at least 2, at least 3, at least 4, at least 5, or at least 6 genes associated with fibrosis and/or inflammation, such as Col1a1, Col3a1, Mmp2, Lgals3, Cd68, and Ccr2. Also provided herein are combinations of the FXR agonist (such as the compound of Formula (I) or a pharmaceutically acceptable salt thereof) and the THRβ agonist (such as the compounds of Formula (II) or a pharmaceutically acceptable salt thereof) for use in treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, using the methods as described herein.

In some embodiments, provided are methods of reducing hepatic inflammation in a patient in need thereof, comprising administering to the patient a combination of the FXR agonist (such as the compound of Formula (I) or a pharmaceutically acceptable salt thereof) and the THRβ agonist (such as the compound of (II) or a pharmaceutically acceptable salt thereof). In some embodiments, the method does not increase LDL-C levels in the patient. In some embodiments, the method decreases LDL-C levels in the patient. In some embodiments, the patient has a disease characterized by liver inflammation. In some embodiments, the patient has liver fibrosis. In some embodiments, the patient has NASH.

In some embodiments, provided are methods of treating a disease characterized by fibrosis of the liver in a patient in need thereof, comprising administering to the patient a combination of the FXR agonist (such as the compound of Formula (I) or a pharmaceutically acceptable salt thereof) and the THRβ agonist (such as the compound of (II) or a pharmaceutically acceptable salt thereof). In some embodiments, the disease is associated with hepatic inflammation. In some embodiments, the method reduces expression of at least one of Col1a1, Col3a1, Mmp2, Lgals3, Cd68, or Ccr2. In some embodiments, the patient has NASH.

In some embodiments, provided are methods of inhibiting expression of genes responsible for the production of collagen in the extracellular matrix of the liver in a patient in need thereof, comprising administering to the patient a combination of the FXR agonist (such as the compound of Formula (I) or a pharmaceutically acceptable salt thereof) and the THRβ agonist (such as the compound of (II) or a pharmaceutically acceptable salt thereof). In some embodiments, the genes are fibroblast genes. In some embodiments, the genes are selected from Col1a1, Col3a1, and Lgals3. In some embodiments, the patient has liver fibrosis. In some embodiments, the patient has NASH.

It is to be understood that recitation of any gene as described herein comprises a reference to orthologs from all species, including humans and rodents.

Also provided herein are uses of the combinations of the FXR agonist (such as the compound of Formula (I) or a pharmaceutically acceptable salt thereof) and the THRβ agonist (such as the compounds of Formula (II) or a pharmaceutically acceptable salt thereof) for manufacture of a medicament for treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, using the methods as described herein.

In some embodiments of the foregoing, the FXR agonist (such as the compound of Formula (I) or a pharmaceutically acceptable salt thereof) is administered orally. In some embodiments of the foregoing, the THRβ agonist (such as the compounds of Formula (II) or a pharmaceutically acceptable salt thereof) is administered orally.

### Articles of Manufacture and Kits

The present disclosure further provides articles of manufacture comprising a compound described herein, or a salt thereof, a composition described herein, or one or more unit dosages described herein in suitable packaging. In certain embodiments, the article of manufacture is for use in any of the methods described herein. Suitable packaging (e.g., containers) is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises at least two compounds described herein, or a pharmaceutically acceptable salt thereof, or a composition comprising a compound described herein, or a pharmaceutically acceptable salt thereof. The kits may employ any of the compounds disclosed herein or a pharmaceutically acceptable salt thereof. In some embodiments, the kit employs an FXR agonist (such as the compound of Formula (I) or a pharmaceutically acceptable salt thereof) and a THRβ agonist (such as the compound of (II) or a pharmaceutically acceptable salt thereof) described herein. The kits may be used for any one or more of the uses described herein, and, accordingly, may contain instructions for the treatment as described herein.

Kits generally comprise suitable packaging. The kits may comprise one or more containers comprising any compound described herein or a pharmaceutically acceptable salt thereof. Each component can be packaged in separate containers or some components can be combined in one container where cross-reactivity and shelf life permit. In some embodiments, the kit includes a container comprising the FXR agonist (such as the compound of Formula (I) or a pharmaceutically acceptable salt thereof) and the THRβ agonist (such as the compound of (II) or a pharmaceutically acceptable salt thereof). In other embodiments, the kit includes a first container comprising FXR agonist (such as the compound of Formula (I) or a pharmaceutically acceptable salt thereof) and a second container comprising the THRβ agonist (such as the compound of (II) or a pharmaceutically acceptable salt thereof).

The kits may be in unit dosage forms, bulk packages (e.g., multi-dose packages) or sub-unit doses. For example, kits may be provided that contain sufficient dosages of a compound as disclosed herein, or a pharmaceutically acceptable salt thereof, and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### Enumerated Embodiments

Embodiment 1. A method of treating a liver disorder in a patient in need thereof, comprising administering to the patient a Farnesoid X Receptor (FXR) agonist and a THRβ agonist, wherein the liver disorder is selected from the group consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).
Embodiment 2. The method of embodiment 1, wherein the FXR agonist is obeticholic acid, cilofexor, tropifexor, EYP001 (Vonafexor, proposed INN), MET409 (Metacrine), or EDP-305 (by Enanta).
Embodiment 3. The method of embodiment 1 or 2, wherein the THRβ agonist is resmetirom (MGL-3196), VK2809 (by Viking Therapeutics), sobetirome, eprotirome, CNPT-101101, CNPT-101207, or ALG-055009 (by Aligo).
Embodiment 4. The method of embodiment 1, wherein the FXR agonist is a compound of formula (I)

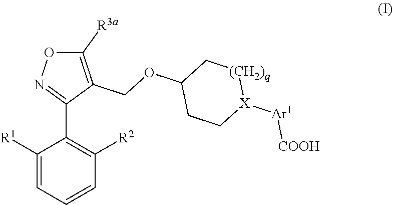

wherein:
q is 1 or 2;
R¹ is chloro, fluoro, or trifluoromethoxy;
R² is hydrogen, chloro, fluoro, or trifluoromethoxy;
R^{3a} is trifluoromethyl, cyclopropyl, or isopropyl;
X is CH or N,
provided that when X is CH, q is 1; and
Ar¹ is indolyl, benzothienyl, naphthyl, phenyl, benzoisothiazolyl, indazolyl, or pyridinyl, each of which is optionally substituted with methyl or phenyl,
or a pharmaceutically acceptable salt thereof.
Embodiment 5. The method of embodiment 4, wherein:
R¹ is chloro or trifluoromethoxy; and
R² is hydrogen or chloro.
Embodiment 6. The method of embodiment 4 or 5, wherein:
R^{3a} is cyclopropyl or isopropyl.
Embodiment 7. The method of any one of embodiments 4 to 6, wherein:
Ar¹ is 5-benzothienyl, 6-benzothienyl, 5-indolyl, 6-indolyl, or 4-phenyl, each of which is optionally substituted with methyl.
Embodiment 8. The method of any one of embodiments 4 to 7, wherein:
q is 1; and
X is N.
Embodiment 9. The method of any one of embodiments 1 and 4 to 8, wherein the FXR agonist is:

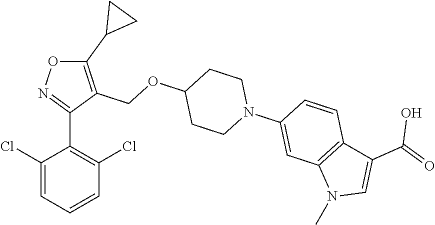 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof.
Embodiment 10. The method of any one of embodiments 1, 2, and 4 to 9, wherein the THRβ agonist is a compound of formula (II)

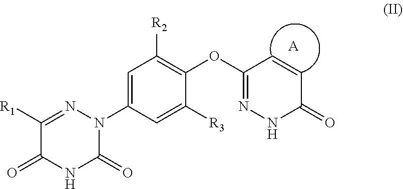

wherein:

R₁ is selected from the group consisting of hydrogen, cyano, substituted or unsubstituted C₁₋₆ alkyl, and substituted or unsubstituted C₃₋₆ cycloalkyl, the substituent being selected from the group consisting of halogen atoms, hydroxy, and C₁₋₆ alkoxy;

R₂ and R₃ are each independently selected from the group consisting of halogen atoms and substituted or unsubstituted C₁₋₆ alkyl, the substituent being selected from the group consisting of halogen atoms, hydroxy, and C₁₋₆ alkoxy;

ring A is a substituted or unsubstituted saturated or unsaturated C₅₋₁₀ aliphatic ring, or a substituted or unsubstituted C₅₋₁₀ aromatic ring, the substituent being one or more substances selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl, and when two substituents are contained, the two substituents can form a ring structure together with the carbon connected thereto; and

the halogen atoms are selected from the group consisting of F, Cl and Br,

or a pharmaceutically acceptable salt thereof.
Embodiment 11. The method of embodiment 10, wherein the THRβ agonist is a compound of formula (IIa)

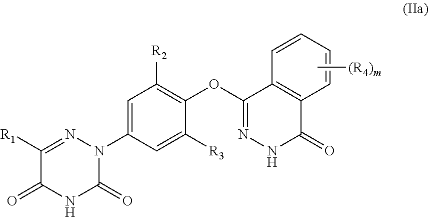

wherein:

R₁ to R₃ are defined as described in claim 10;

R₄ is selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl;

m is an integer from the range 1 to 4; and

the halogen atoms are selected from the group consisting of F, Cl and Br.

or a pharmaceutically acceptable salt thereof.
Embodiment 12. The method of embodiment 10 or 11, wherein R₄ is selected from the group consisting of hydrogen, halogen atoms, hydroxy, C₁₋₆ alkyl, C₁₋₆ alkoxy and C₃₋₆ cycloalkyl; and

m is an integer from the range 1 to 3.
Embodiment 13. The method of any one of embodiments 10 to 12, wherein R₁ is selected from the group consisting of hydrogen, cyano, and substituted or unsubstituted C₁₋₆ alkyl, the substituent being selected from the group consisting of halogen atoms, hydroxy, and C₁₋₆ alkoxy; and

the halogen atoms are selected from the group consisting of F, Cl and Br.
Embodiment 14. The method of any one of embodiments 1, 2 and 4 to 13, wherein the THRβ agonist is:

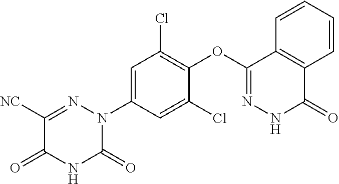 [SMILES: N#Cc1nn(-c2cc(Cl)c(Oc3n[nH]c(=O)c4ccccc34)c(Cl)c2)c(=O)[nH]c1=O]

or a pharmaceutically acceptable salt thereof.
Embodiment 15. The method of any one of embodiments 1 to 14, wherein the FXR agonist and the THRβ agonist are administered simultaneously.
Embodiment 16. The method of any one of embodiments 1 to 14, wherein the FXR agonist and the THRβ agonist are administered sequentially.
Embodiment 17. The method of any one of embodiments 1 to 16, wherein the administration does not result in pruritus in the patient at a severity of Grade 2 or more.
Embodiment 18. The method of any one of embodiments 1 to 17, wherein the administration does not result in pruritus in the patient at a severity of Grade 1 or more.
Embodiment 19. The method of any one of embodiments 1 to 18, wherein the administration does not result in pruritus in the patient.
Embodiment 20. The method of any one of embodiments 1 to 19, wherein the patient also has diabetes mellitus and/or a cardiovascular disorder.
Embodiment 21. The method of any one of embodiments 1 to 20, wherein the treatment period is the remaining lifespan of the patient.
Embodiment 22. The method of any one of embodiments 1 to 21, wherein the method does not comprise administering an antihistamine, an immunosuppressant, a steroid, rifampicin, an opioid antagonist, or a selective serotonin reuptake inhibitor (SSRI).
Embodiment 23. The method of any one of embodiments 1 to 22, wherein the FXR agonist is administered once daily or twice daily.
Embodiment 24. The method of any one of embodiments 1 to 23, wherein the THRβ agonist is administered once daily or twice daily.
Embodiment 25. The method of any one of embodiments 1 to 24, wherein the administration comprises administering the FXR agonist daily for a treatment period of one or more weeks.
Embodiment 26. The method of any one of embodiments 1 to 25, wherein the administration comprises administering the THRβ agonist daily for a treatment period of one or more weeks.
Embodiment 27. The method of any one of embodiments 1 to 26, wherein the liver disorder is selected from the group consisting of non-alcoholic fatty liver disease (NAFLD) and non-alcoholic steatohepatitis (NASH).
Embodiment 28. The method of any one of embodiments 1-26, wherein the liver disorder is non-alcoholic steatohepatitis.
Embodiment 29. A pharmaceutical composition comprising an therapeutically effective amount of an FXR agonist, a therapeutically effective amount of a THRβ agonist, and a pharmaceutically acceptable carrier, diluent, excipient, or a combination of any of the foregoing.
Embodiment 30. A dosage form comprising a therapeutically effective amount of an FXR agonist and a therapeutically effective amount of a THRβ agonist.
Embodiment 31. A kit comprising a container comprising an FXR agonist and a THRβ agonist.
Embodiment 32. A kit comprising a first container comprising an FXR agonist and a second container comprising a THRβ agonist.
Embodiment 33. The pharmaceutical composition of embodiment 29, the dosage form of embodiment 30, or the kit of embodiment 31 or 32, wherein the FXR agonist is

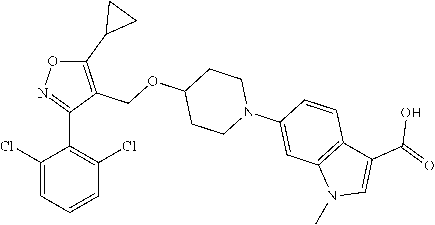 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, and the THRβ agonist is:

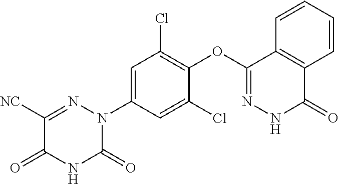 [SMILES: N#Cc1nn(-c2cc(Cl)c(Oc3n[nH]c(=O)c4ccccc34)c(Cl)c2)c(=O)[nH]c1=O]

or a pharmaceutically acceptable salt thereof.

### EXAMPLES

The combination treatment provided herein can be tested by administering the combination of the agents to a well-known mouse model and evaluating the results. Methods of such testing can be adapted from those known. See, e.g., US Pat. Pub. No. 2015/0342943, incorporated herein by reference.

### Example 1: In Vitro Metabolic Stability

The rate of hepatic metabolism of Compound 1 was assessed in cryopreserved hepatocytes to determine the in vitro half-life of the compound. 1 μM of Compound 1 was mixed with preconditioned mouse, rat, dog, monkey, or human hepatocytes (0.5×10⁶ cells/mL) and allowed to incubate at 37° C. for 2 hours, with samples collected at several time points and assayed for Compound 1. In vitro half-life values were determined and scaled to predict hepatic clearance (CL_{pred}) and hepatic extraction using the well-stirred liver model with no correction for plasma protein as described in Obach et al., The Prediction of Human Pharmacokinetic Parameters from Preclinical and In Vitro Metabolism Data, J. of Pharmacology and Experimental Therapeutics, vol. 283, no. 1, pp. 46-58 (1997). Results are shown in Table 1, which demonstrate that Compound 1 was moderately metabolized in hepatocytes of all tested species.

TABLE 1
In Vitro metabolic stability of Compound 1

| | | | Hepatic |
|---|---|---|---|
| | t_{1/2} | In vitro Metabolic | Extraction |
| Species | (min) | CL_{pred} (L/h/kg) | (%) |
| Mouse | 43.6 ± 2.83 | 4.36 ± 0.06 | 80.7 ± 1.02 |
| Sprague- | 131 ± 4.11 | 1.57 ± 0.03 | 47.3 ± 0.78 |
| Dawley Rat | | | |
| Beagle Dog | 126 ± 15.5 | 1.32 ± 0.05 | 71.0 ± 2.49 |
| Cynomolgus | 63.4 ± 0.78 | 1.68 ± 0.01 | 64.4 ± 0.28 |
| Monkey | | | |
| Human | 84.1 ± 6.48 | 0.83 ± 0.22 | 67.0 ± 1.73 |

### Example 2: In Vitro OATP Transport Assay

A polarized monolayer of MDCK-II cells grown on a permeable support was used to test the ability of organic-anion-transporting polypeptide (OATP) 1B1 or OATP 1B3 to transport Compound 1 across the lipid bilayer and into the cells. The MDCK-II cells were transfected one of (1) a vector to express OATP 1B1, (2) a vector to express OATP 1B3, or (3) a control vector. Expression was induced in the cells before culturing the cells at 37° C. in 5% CO₂ atmosphere. After inducing expression, the cells were treated with 1 μM, 3 μM, and 10 μM Compound 1, or 3 μM Compound 1 and 100 μM rifampin. Cellular uptake of Compound 1 was then measured. Results from this experiment demonstrated that Compound 1 is not an OATP 1B1 or OATP 1B3 substrate.

### Example 3: Pharmacokinetics Assay

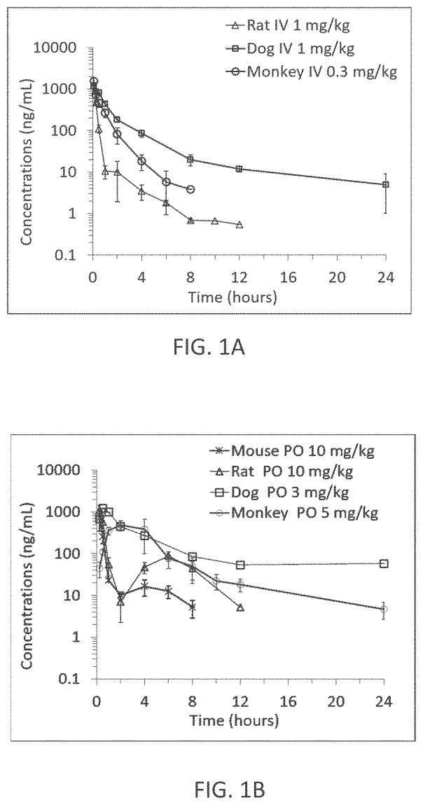

Compound 1 was administered to Sprague-Dawley (SD) rats intravenously at 1 mg/kg (n=3) or orally at 10 mg/kg (n=3), to beagle dogs intravenously at 1 mg/kg (n=3) or orally at 3 mg/kg (n=3), to cynomolgus monkeys intravenously at 0.3 mg/kg (n=6) or orally at 5 mg/kg (n=6), and to mice orally at 5 mg/kg (n=9). Compound 1 for oral administration to SD rats was formulated in a vehicle containing 10% DMSO, 10% Cremophor-EL, and 80% aqueous solution (10% 2-hydroxypropyl-β-cyclodextrin). Compound 1 for oral administration to beagle dogs was formulated with an aqueous solution containing 1% carboxymethyl cellulose, 0.25% Tween-80, and 0.05% antifoam. Compound 1 for oral administration to cynomolgus monkeys was formulated with 10% Solutol, 20% PEG400, 0.5% Tween-80 and 69.5% deionized water. Serial blood samples were collected, and plasma concentrations of the Compound 1 were measured. Results are shown in FIG. 1A (IV administration) and FIG. 1B (oral administration), and in Table 2. The results demonstrate that Compound 1 has low to moderate clearance in vivo. The volume of distribution (V_{dss}) of Compound 1 is greater than the volume of total body water (0.70 L/kg) in rat and dog. Smaller V_{dss} in monkeys is correlated with higher plasma protein binding.

TABLE 2
Pharmacokinetic parameters of Compound 1

| | CL | | IV Terminal | Oral |
|---|---|---|---|---|
| Species | (L/h/kg) | V_{dss} (L/kg) | t_{1/2} (h) | Bioavailability (%) |
| Sprague- | 2.55 | 1.31 | 2.45 | 21 |
| Dawley Rat | | | | |
| Beagle Dog | 0.54 | 1.92 | 5.67 | 82 |
| Cynomolgus | 0.30 | 0.6 | 1.32 | 18 |
| Monkey | | | | |

### Example 4: Tissue Distribution of Compound 1

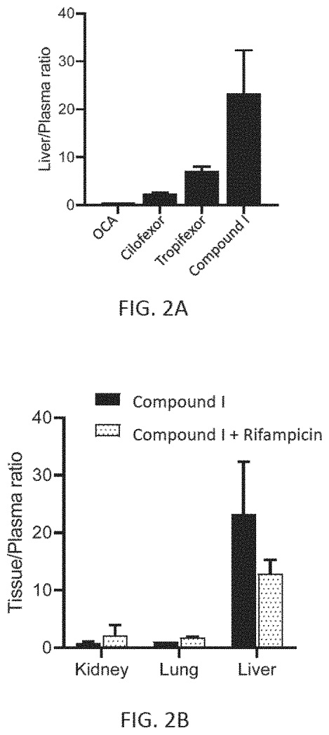

Tissue distribution of Compound 1 administered to rats was determined and compared to distribution other Farnesoid X Receptor (FXR) agonists cilofexor, tropifexor, and obeticholic acid (OCA). The tested compounds were administered to SD rats (n=3 per compound) by way of 30 minute intravenous infusion at 2 mg/kg. Blood, liver, kidney, and lung tissue samples were collected from the rats to determine a tissue/plasma ratio. The liver tissue/plasma ratio for the compounds is shown in FIG. 2A, which demonstrates that substantially more of Compound 1 localizes to the liver tissue compared to the other tested compounds. Co-administration of Compound 1 with 100 μM rifampin does not result in a significant change in distribution of Compound 1 to the liver (FIG. 2B). These results collectively demonstrated that Compound 1 is preferentially distributed to the liver and exhibited high liver/plasma ratio in rodent species, approximately 3 to 20-fld higher than other FXR agonists being studied for the treatment of NASH (cilofexor, tropifexor, and OCA).

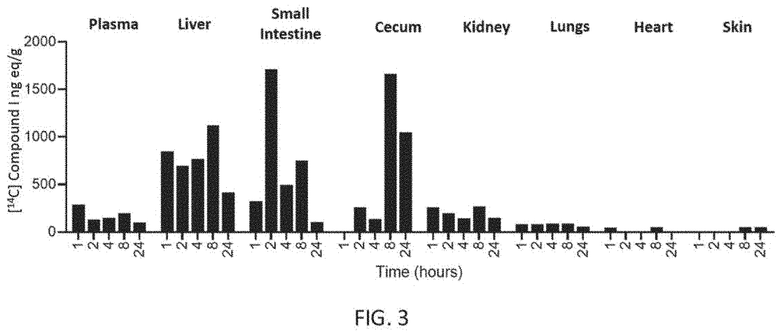

Radiolabeled (¹⁴C) Compound 1 was also administered to Long-Evans rats at an oral dose of 5 mg/kg (100 μCi/kg). Plasma, liver, small intestine, cecum, kidney, lung, heart and skin tissue samples were collected up to 168 hours, and the amount of radioactive material at various time points was measured. Results are shown in FIG. 3. Liver, small intestine, and cecum had the most radioactive material.

### Example 5: Metabolism of Compound 1

Radiolabeled (¹⁴C) Compound 1 was administered to bile duct intact or cannulated SD rats orally at 5 mg/kg or intravenously at 2 mg/kg (n=3 for each of the four cohorts) for a total radioactive dose of 100 μCi/kg. Blood, bile, feces, and urine samples were collected from each rat for up to 168 hours. Compound 1 was metabolized into an acyl glucuronide metabolite prior to biliary excretion, which was determined as the major elimination pathway for the compound.

### Example 6: Pharmacokinetics/Pharmacodynamics Profile

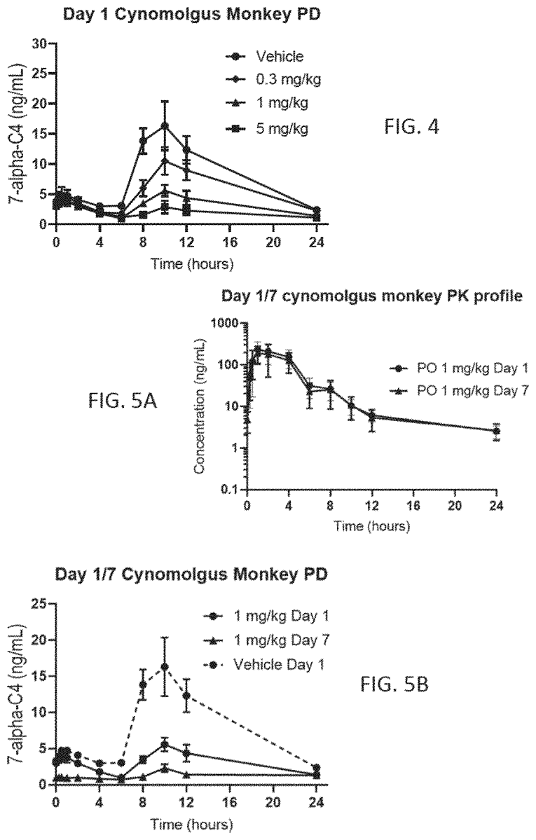

Pharmacokinetics/pharmacodynamics (PK/PD) profiles for cynomolgus monkeys was determined by administering an oral dose of Compound 1 suspension at doses of 0 (vehicle), 0.3, 1, or 5 mg/kg, and collecting blood samples for up to 24 hours. The pharmacodynamics were measured as a function of 7-alpha-hydroxy-4-cholesten-3-one (7AC4) reduction (FIG. 4), as quantified by LC-MS/MS. Pharmacokinetics data is presented in Table 3, and were determined by non-compartmental analysis.

TABLE 3
Pharmacokinetic parameters of Compound 1

| | PK Parameters | | |
|---|---|---|---|
| Compound 1 | AUC₀₋₂₄ | Cₘₐₓ | |
| dose | (ng*hr/mL) | (ng/mL) | Tₘₐₓ (hr) |
| 0.3 mg/kg | 196 ± 64 | 58.8 ± 30.2 | 2.17 ± 1.47 |
| 1 mg/kg | 1000 ± 419 | 257 ± 124 | 1.83 ± 1.17 |
| 5 mg/kg | 2720 ± 1500 | 709 ± 458 | 2.25 ± 1.47 |

Compound 1 was also orally administered at 1 mg/kg for 7 consecutive days to cynomolgus monkeys (n=6) to determine the PK/PD profile following multiple doses. Results of this study are shown in FIG. 5A (PK profile) and FIG. 5B (PD profile) and Table 4, and demonstrate that the plasma exposure of Compound 1 was comparable on day 1 and day 7 and that sustained suppression of the pharmacodynamics biomarker 7AC4 was achieved after repeated oral dosing.

TABLE 4
Pharmacokinetic parameters of Compound 1

| PK | Cₘₐₓ | AUC₀₋₂₄ | |
|---|---|---|---|
| Parameters | (ng/mL) | (ng*hr/mL) | Tₘₐₓ (hr) |
| Day 1 | 257 ± 124 | 1000 ± 419 | 1.83 ± 1.17 |
| Day 7 | 221 ± 121 | 858 ± 425 | 1.25 ± 0.61 |

### Example 7: Mechanism of Action

C57BL/6 mice were administered a single oral dose of 10 mg/kg Compound 1 (n=6), 30 mg/kg OCA (n=6), or a vehicle control (n=6), and tissue RNA samples were collected 6 hours after dose administration. The RNA was analyzed by RT-qPCR and RNAseq.

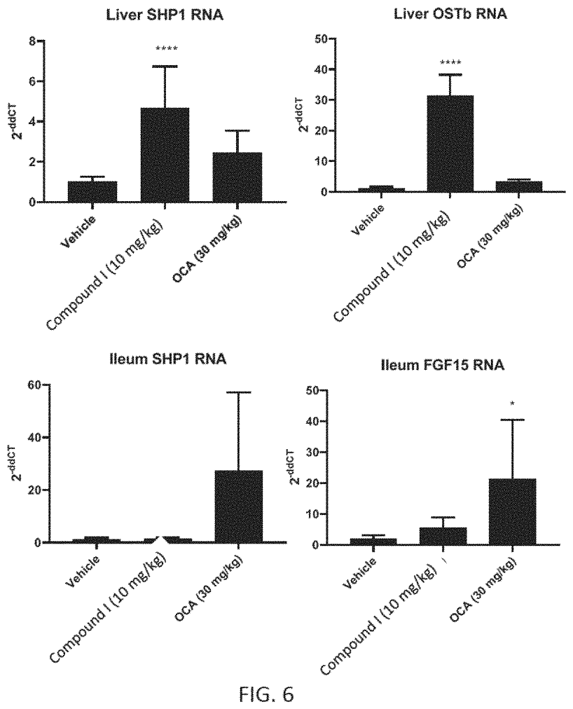

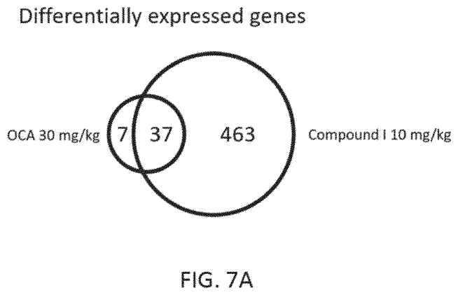

For RT-qPCR, gene-specific primers were used to quantitate FXR-regulated gene expression in liver and ileum using the 2-ddCT method. Results are shown in FIG. 6 (data presented as mean±SEM; **** indicates p<0.0001 and * indicates p<0.05 versus vehicle, with statistics determined by one-way ANOVA followed by Tukey). This data indicates that Compound 1 preferentially induces FXR-specific genes in the liver of mice.

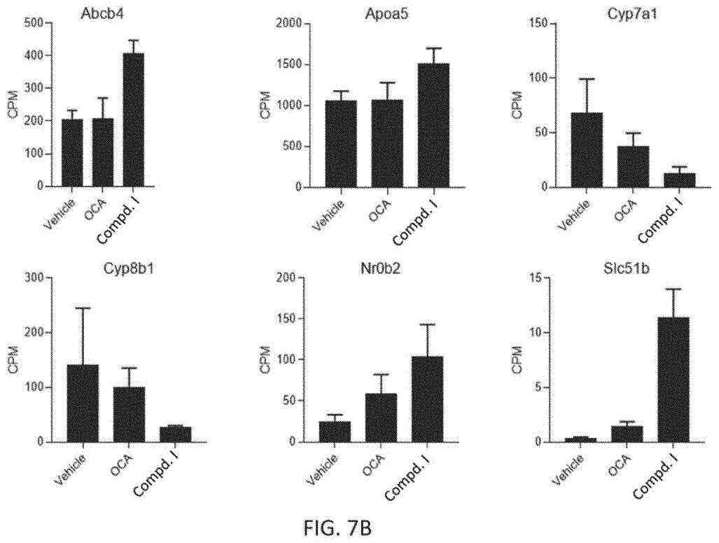

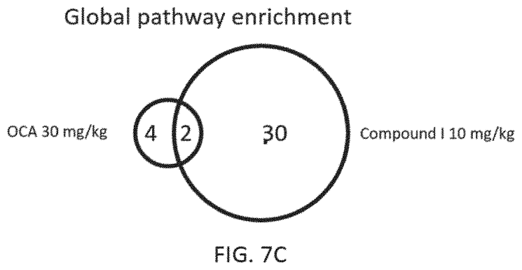

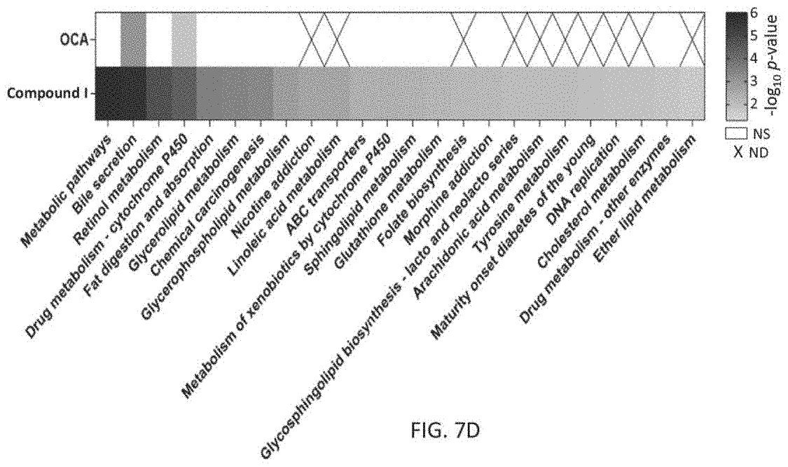

For RNAseq analysis, mRNA was extracted from total liver and sequenced using standard Illumina library preparation and sequencing protocols. Differentially expressed genes (DEG) were determined using RSEM and edgeR software packages and analyzed using Advaita Bio's iPathwayGuide software. Results are shown in FIG. 7A-7D, which indicate that Compound 1 modulates a significantly higher number of genes and metabolic pathways relevant to NASH compared to OCA. FIG. 7A shows that administration of Compound 1 modulates expression of 500 NASH-related genes, OCA modulates expression of 44 NASH-related genes, including 37 common NASH-related genes modulated by both Compound 1 and OCA, relative to vehicle control (fold change ≥1.5; q-value <0.05). FIG. 7B shows average expression levels (as shown by CPM value) of select FXR-related genes in vehicle, OCA, and Compound 1 treated mice. FIG. 7C shows that administration of Compound 1 causes enrichment of 32 global pathways and that administration of OCA causes enrichment of 6 global pathways, including 2 common global pathways to both Compound 1 and OCA administration. FIG. 7D shows the 25 pathways most statistically enriched upon Compound 1 administration, and compares the enrichment of those pathways to the enrichment upon OCA administration. Overall, RNAseq analysis of livers from mice treated with Compound 1 showed a more robust modulation of FXR-related genes and metabolic pathways relevant to non-alcoholic fatty liver disease compared to OCA treatment.

### Example 8: Clinical Study

First Study. Heathy human volunteer subjects were orally dosed on a daily basis with Compound 1 at 5 mg (n=9), 75 mg (n=9), 200 mg, or 400 mg (n=18), or received a placebo (n=12) for 14 days. During this study, no incidences of pruritus were observed.

Second Study. Compound 1 was administered daily for 7 days at oral doses of 25 mg (n=11), 75 mg (n=10), or 150 mg (n=10), or received a placebo (n=5) to human subjects. 7-alpha-hydroxy-4-cholesten-3-one (7AC4) levels in the patients were periodically measured, as shown in Table 5, which indicated that levels were suppressed by Compound 1. In a separate study published by an independent group, FXR agonist MET409 (Metacrine) was reportedly administered daily to healthy human volunteers at doses of 20 mg 40 mg, 50 mg, 80 mg, 100 mg, or 150 mg, and 7AC4 levels measured as shown in Table 5. See Chen et al., MET409, an Optimized Sustained FXR Agonist, Was Safe and Well-Tolerated in a 14-Day Phase 1 Study in Healthy Subjects, The International Liver Congress, Vienna, Austria, Apr. 10-14, 2019. While pruritus was observed in subjects receiving MET409 at doses of 100 mg or greater, no pruritus was observed for subjects taking the highest doses of Compound 1. Other FXR agonists, such as cilofexor, tropifexor, OCA, ED-305 (Enanta) are all known to result in pruritus in longer term studies.

TABLE 5
Comparison of MET409 and Compound 1

| | MET409 | | | | | |
|---|---|---|---|---|---|---|
| | 50 mg | 80 mg | 100 mg | Compound 1 | | |
| Parameters | MET409 | MET409 | MET409 | 25 mg | 75 mg | 150 mg |
| AUC | 6404 | 12479 | 16519 | 645 | 1480 | 2164 |
| ng*h/ml | | | | | | |
| % 7AC4 | 85% | 96% | 99% | 75% | 82% | 93% |
| suppression | | | | | | |
| at nadir | | | | | | |
| AUC/% | 75 | 130 | 166 | 8.6 | 18 | 23 |
| 7AC4 ratio | | | | | | |
| Pruritus | No | No | Yes | No | No | No |

### Example 9: Mouse Model of NASH

The effect of Compound 1 on NASH was assessed using a mouse model, in which NASH is induced by a high fat diet in combination with CC1₄ administration.

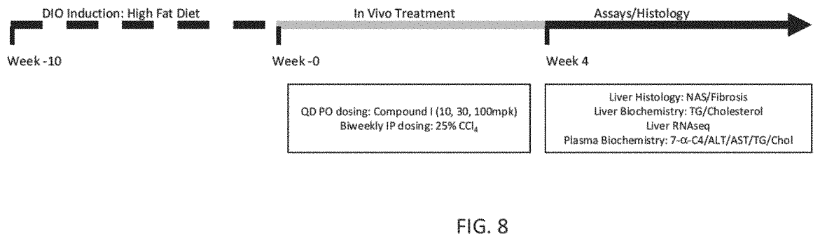

Mice C57/BL6J mice were fed a high fat diet (D12492, Research Diet, fat/protein/carbohydrate 60/20/20 Kcal %, 10 w) to induce obesity (>36g mouse) prior to daily oral Compound 1 and biweekly intraperitoneal carbon tetrachloride (CCl₄) treatment for four weeks. FIG. 8. Compound 1 was administered at a dose of 10, 30, and 100 mg/kg.

Following 28 days of Compound 1 dosing, serum lipids, serum transaminases and liver lipids were analyzed. Hematoxylin & Eosin (H&E) and Sirius Red histological staining of liver tissue was used to quantitate NAFLD activity score (NAS), steatosis, ballooning, inflammation and fibrosis. Plasma 7-alpha-hydroxy-4-cholesten-3-one (7AC4) was measured as a biomarker of FXR activation. Gene expression of RNA was analyzed by RT-qPCR and RNAseq.

Nonalcoholic Fatty Liver Disease Activity Score (NAS) is a composite score used to assess NASH. NAS is calculated based upon liver steatosis, inflammation, and ballooning and was determined by analysis of liver tissue histology using H&E stain. Specifically, inflammation score was calculated based upon H&E staining: Score 0, none; 1, <2 foci per 200× field; 2, 2-4 foci per 200× field; 3, >4 foci per 200× field. Steatosis score was calculated by H&E staining as follows: Score 0, <5%; 1,5-33%; 2, >33-66%; 3, >66%). Hepatocellular ballooning is a form of liver cell injury associated with cell swelling and is also measured by H&E stained liver sections. The ballooning score is calculated as follows: 0-no hepatocyte ballooning; 1-few ballooning hepatocytes; 2-many hepatocytes with prominent ballooning.

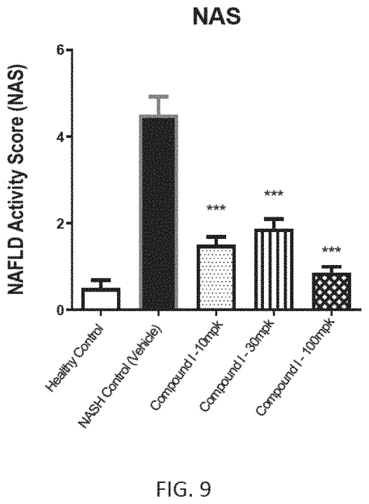

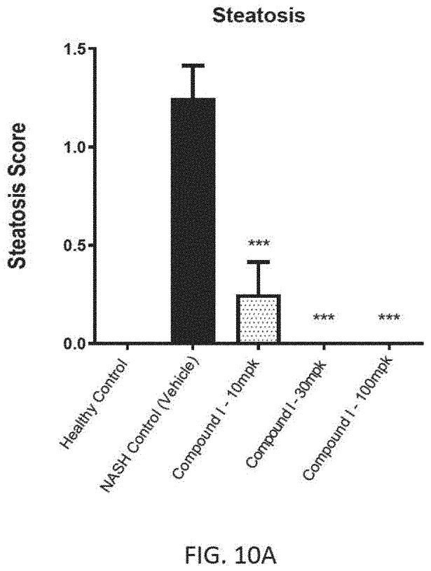

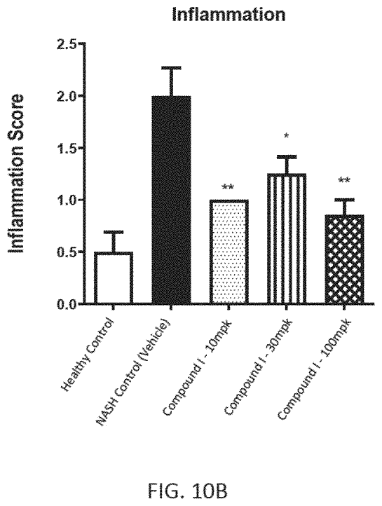

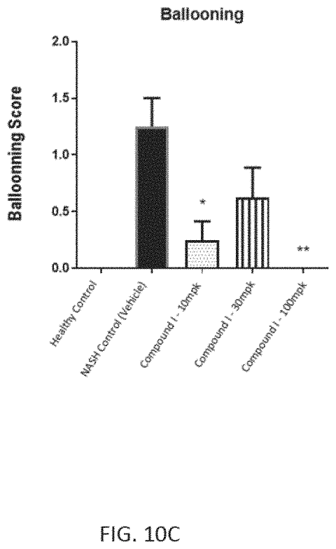

As shown in FIG. 9, mice treated with 10, 30, or 100 mg/kg Compound 1 had a significantly lower NAS score as compared to untreated NASH mice. Treatment with Compound 1 also significantly reduced steatosis, inflammation and ballooning compared to untreated NASH mice. FIG. 10A-C.

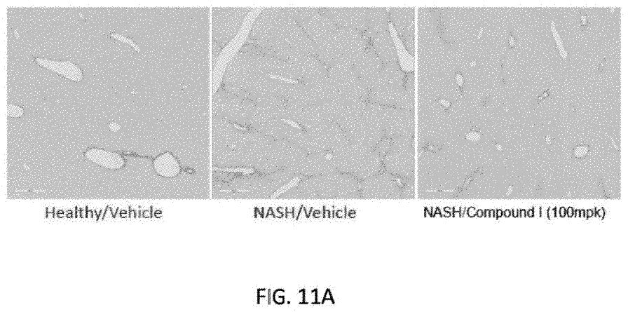

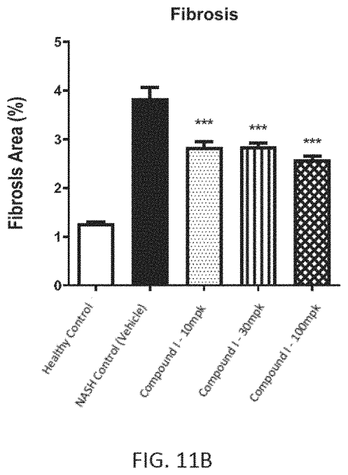

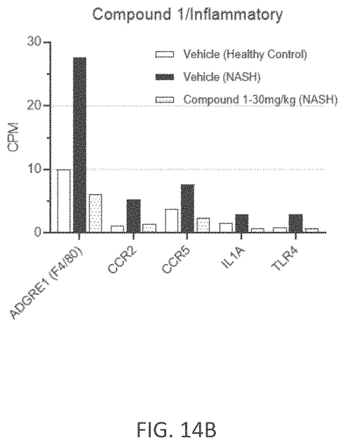

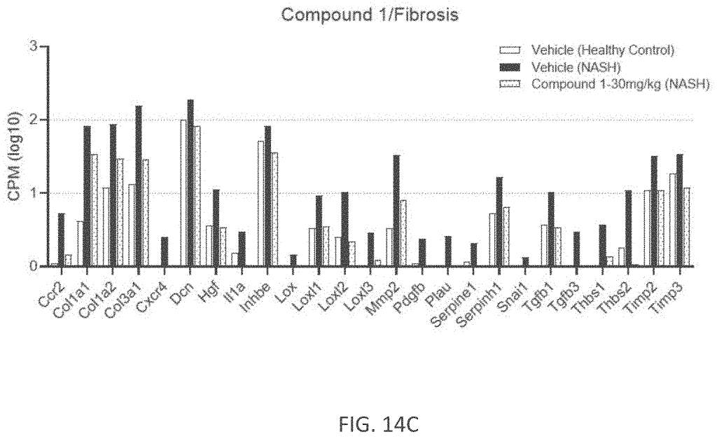

Liver fibrosis was quantified by histological analysis of the percentage of Sirius Red-positive liver sections. FIG. 11A shows representative histology for healthy mice, NASH mice, and NASH mice treated with Compound 1 at 100 mg/kg. FIG. 11B shows quantification of the fibrosis area of mice treated with Compound 1. Treatment with 10, 30 or 100 mg/kg Compound 1 resulted in statistically significant reduced fibrosis compared to untreated NASH control. As shown in FIG. 14A, Compound 1 administered at 10, 30, or 100 mg/kg resulted in decreased collagen, type 1, alpha 1 expression in the liver as compared to control NASH mice.

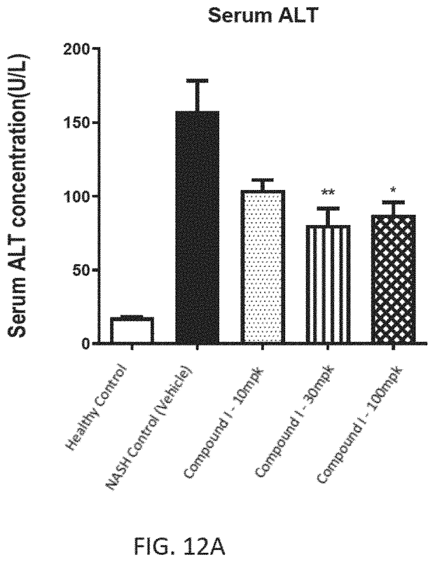

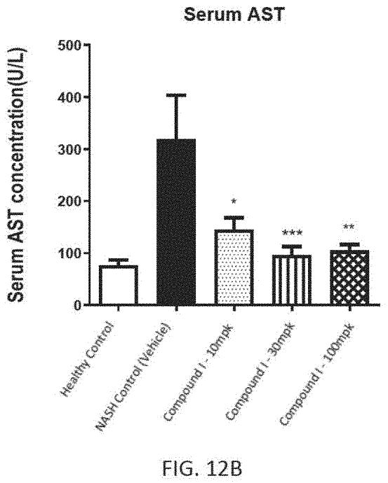

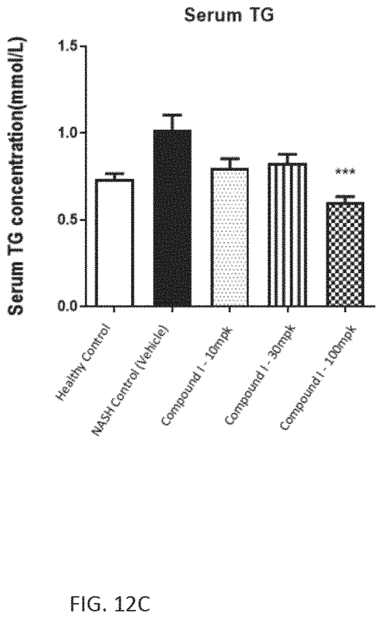

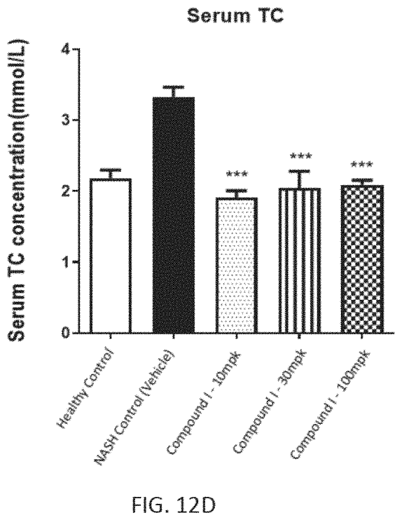

After treatment, serum was analyzed for alanine amino transferase (ALT), aspartate amino transferase (AST), triglyceride, and total cholesterol levels. As shown in FIG. 12A and FIG. 12B serum ALT and AST levels were reduced in mice treated with Compound 1. FIG. 12C shows a statically significant reduction in serum triglyceride concentration in mice treated with 100 mg/kg Compound 1. FIG. 12D shows statistically significant reduction of total cholesterol level in mice treated with 10, 30, and 100 mg/kg Compound 1.

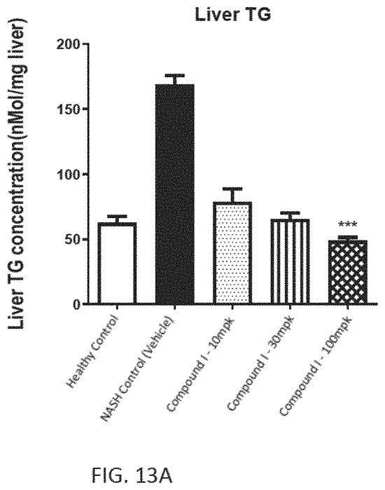

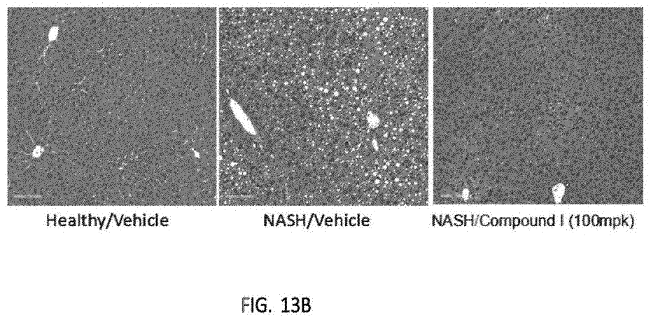

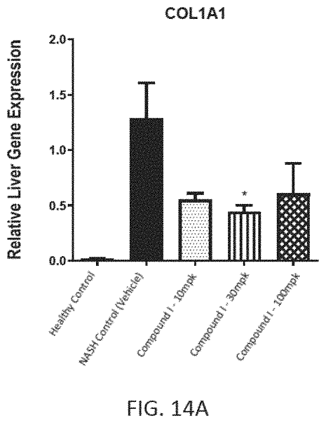

Liver triglycerides were measured from liver tissue using a biochemical analyzer (Hitachi-700). FIG. 13A shows the concentration of liver triglycerides in control mice or mice treated with 10, 30, or 100 mg/kg Compound 1. Mice treated with 100 mg/kg Compound 1 showed statistically significant reduced triglyceride levels. FIG. 13B shows a representative histology section.

The effect of Compound 1 on gene expression was analyzed using RT-qPCR or RNA-seq of liver samples (FIG. 14A-C and Table 6). Table 6 shows the effect of Compound 1 on FXR-regulated gene expression in the liver. The expression level of each indicated gene (as defined by gene count per million (CPM) value) after treatment with Compound 1 was divided by the expression level of that gene in vehicle treated animals to determine the activity of Compound 1 relative to vehicle.

TABLE 6
Expression of FXR-target, inflammatory, and fibrosis genes

| Gene | Compound 1 (30 mg/kg) Relative to Vehicle |
|---|---|
| SHP | 4.6 |
| BSEP | 5.1 |
| OST-B | 135.7 |
| CYP7A1 | 0.02 |
| CYP8B1 | 0.007 |

EC₅₀ concentration of Compound 1 for FXR was determined by a fluorescence-based FXR coactivation assay. Half-log serial dilutions of Compound 1 or OCA (obeticholic acid, a known FXR agonist) (10 μM-3 nM) were incubated with human FXR ligand binding domain produced in Sf9 insect cells, labeled coactivator SRC-1 peptide and TR-FRET Coregulator Buffer G for 1 h at 25° C. TGR5 activity was measured using a cell-based cAMP assay. See Kawamata et al JBC 278 (11)935-440 (2003). Half-log serial dilutions of Compound 1 or OCA (10 μM-3 nM) were added to Chinese Hamster Ovary cells expressing recombinant human TGR5. After 30 min at RT, cAMP was measured using an HTRF readout. EC₅₀ values for FXR-regulated gene expression were determined using a cell-based RNA assay. Half-log serial dilutions of Compound 1 or OCA (3 μM-3 nM) were added to human HuH7 hepatoma cells. After 11 h at 37° C., RNA was isolated and analyzed by RT-qPCR using primers to FXR-related genes: small heterodimer partner (SHP), bile salt export pump (BSEP) and fibroblast growth factor 19 (FGF-19).

As shown in Table 7, Compound 1 is a potent and selective FXR agonist.

TABLE 7
EC₅₀ of Compound 1

| | EC₅₀ of | OCA |
|---|---|---|
| Assay | Compound 1 (nM) | EC₅₀ (nM) |
| FXR Agonist | 57 | 73 |
| TGR5 Agonist | >10,000 | 770 |
| SHP Gene induction/HuH7 | 50 | 200 |
| BSEP Gene induction/HuH7 | 40 | 200 |
| FGF-19 Gene Induction/HuH7 | 40 | 130 |

In summary, Compound 1 is a potent and selective FXR agonist. Compound 1 reduced expression of inflammatory and fibrosis related genes and strongly suppressed liver steatosis, inflammation, ballooning, and fibrosis in a mouse model of NASH.

### Example 10

Exemplary compounds of formula (II) are provided in Table 8 below. Compound 2 is listed in the table as compound number 2.

TABLE 8
Exemplary compound of formula (II)

| Compound | Structure |
|---|---|
| 2 | 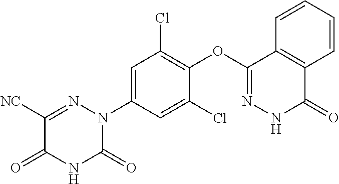 [SMILES: N#Cc1nn(-c2cc(Cl)c(Oc3n[nH]c(=O)c4ccccc34)c(Cl)c2)c(=O)[nH]c1=O] |
| 3 | 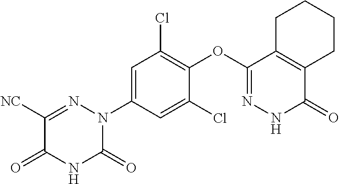 [SMILES: N#Cc1nn(-c2cc(Cl)c(Oc3n[nH]c(=O)c4c3CCCC4)c(Cl)c2)c(=O)[nH]c1=O] |
| 4 | 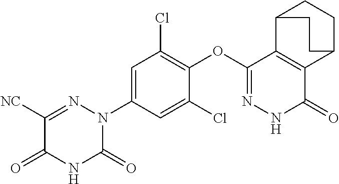 [SMILES: N#Cc1nn(-c2cc(Cl)c(Oc3n[nH]c(=O)c4c3C3CCC4CC3)c(Cl)c2)c(=O)[nH]c1=O] |
| 5 | 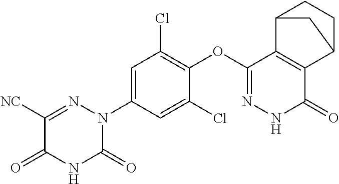 [SMILES: N#Cc1nn(-c2cc(Cl)c(Oc3n[nH]c(=O)c4c3C3CCC4C3)c(Cl)c2)c(=O)[nH]c1=O] |
| 6 | 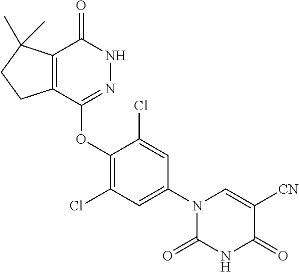 [SMILES: CC1(C)CCc2c(Oc3c(Cl)cc(-n4cc(C#N)c(=O)[nH]c4=O)cc3Cl)n[nH]c(=O)c21] |
| 7 | 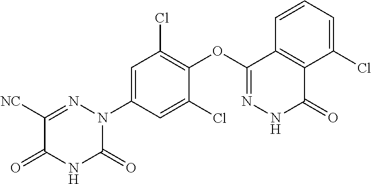 [SMILES: N#Cc1nn(-c2cc(Cl)c(Oc3n[nH]c(=O)c4c(Cl)cccc34)c(Cl)c2)c(=O)[nH]c1=O] |
| 8 | 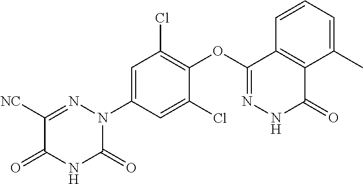 [SMILES: Cc1cccc2c(Oc3c(Cl)cc(-n4nc(C#N)c(=O)[nH]c4=O)cc3Cl)n[nH]c(=O)c12] |
| 9 | 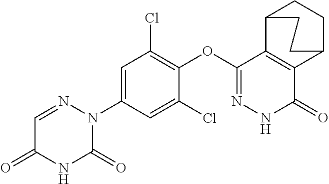 [SMILES: O=c1cnn(-c2cc(Cl)c(Oc3n[nH]c(=O)c4c3C3CCC4CC3)c(Cl)c2)c(=O)[nH]1] |
| 10 | 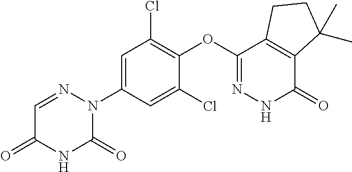 [SMILES: CC1(C)CCc2c(Oc3c(Cl)cc(-n4ncc(=O)[nH]c4=O)cc3Cl)n[nH]c(=O)c21] |
| 11 | 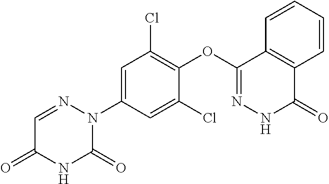 [SMILES: O=c1cnn(-c2cc(Cl)c(Oc3n[nH]c(=O)c4ccccc34)c(Cl)c2)c(=O)[nH]1] |

A compound of formula (II), in some embodiments, is selected from the group consisting of:
2-(3,5-dichloro-4-((4-oxo-3,4,5,6,7,8-hexahydrophthalazin-1-yl)oxy)phenyl)-3,5-dioxo-2,3,4,5-tetrahydro-1,2,4-triazine-6-nitrile;
2-(3,5-dichloro-4-((4-oxo-3,4,5,6,7,8-hexahydro-5,8-ethanophthalazin-1-yl)oxy)phenyl)-3,5-dioxo-2,3,4,5-tetrahydro-1,2,4-triazine-6-nitrile;
2-(3,5-dichloro-4-((4-oxo-3,4,5,6,7,8-hexahydro-5,8-methanophthalazin-1-yl)oxo)phenyl)-3,5-dioxo-2,3,4,5-tetrahydro-1,2,4-triazine-6-nitrile;
1-(3,5-dichloro-4-((7,7-dimethyl-1-oxo-2,5,6,7-tetrahydro-1H-cyclopentane[d]pyridazin-4-yl)oxy)phenyl)-2,4-dioxo-1,2,3,4-tetrahydropyrimidine-5-nitrile;
2-(3,5-dichloro-4-((4-oxo-3,4-dihydrophthalazin-1-yl)oxo)phenyl)-3,5-dioxo-2,3,4,5-tetrahydro-1,2,4-triazine-6-nitrile;
2-(3,5-dichloro-4-((5-chloro-4-oxo-3,4-dihydrophthalazin-1-yl)oxo)phenyl)-3,5-dioxo-2,3,4,5-tetrahydro-1,2,4-triazine-6-nitrile;
2-(3,5-dichloro-4-((5-methyl-4-oxo-3,4-dihydrophthalazin-1-yl)oxo)phenyl)-3,5-dioxo-2,3,4,5-tetrahydro-1,2,4-triazine-6-nitrile;
2-(3,5-dichloro-4-((4-oxo-3,4,5,6,7,8-hexahydro-5,8-ethanophthalazin-1-yl)oxo)phenyl)-1,2,4-triazine-3,5(2H,4H)-dione;
2-(3,5-dichloro-4-((7,7-dimethyl-1-oxo-2,5,6,7-tetrahydro-1H-cyclopentyl[d]pyridazin-4-yl)oxo)phenyl)-1,2,4-triazine-3,5(2H,4H)-dione; and
2-(3,5-dichloro-4-((4-oxo-3,4-dihydrophthalazin-1-yl)oxo)phenyl)-1,2,4-triazine-3,5-(2H,4H)dione.

A compound of formula (II) has a good agonistic activity toward the THRβ receptor, and an improved selectivity toward THRα as compared with Reference compound in the reference documents (“Discovery of 2-[3,5-Dichloro-4-(5-isopropyl-6-oxo-1,6-dihydropyridazin-3-yloxy)phenyl]-3,5-dioxo-2,3,4,5-tetrahydro[1,2,4]triazine-6-carbonitrile (MGL-3196), a Highly Selective Thyroid Hormone Receptor β Agonist in Clinical Trials for the Treatment of Dyslipidemia,” Martha et al., Journal of Medicinal Chemistry, 2014, 3912-3923). The structure of the reference compound is

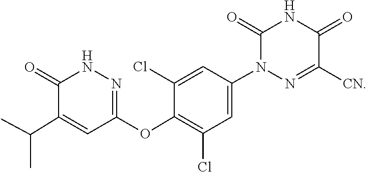 [SMILES: CC(C)c1cc(Oc2c(Cl)cc(-n3nc(C#N)c(=O)[nH]c3=O)cc2Cl)n[nH]c1=O]

Test data are shown in Table 9 and Table 10.

TABLE 9
Binding activity of compounds to the thyroxine receptor beta

| | IC50 | | THRα/β |
|---|---|---|---|
| | THRβ binding force | THRα binding force | selectivity |
| Compound | (μM) | (μM) | (factor) |
| 2 | 0.17 | >10 | >58.8 |
| 3 | 1.23 | >10 | >8.1 |
| 4 | 2.33 | >10 | >4.29 |
| 5 | 5.2 | >10 | >1.92 |
| 6 | 0.36 | 4.3 | >11.9 |
| 7 | 1.47 | >10 | >6.80 |
| 8 | 1.78 | >10 | 5.61 |
| 9 | 0.80 | 0.2 | 0.25 |
| 10 | 0.17 | 1.22 | 7.17 |
| 11 | 0.262 | | |
| Reference | 0.26 | 5.0 | 19.2 |
| compound | | | |
| triiodothyronine | 0.00052 | 0.00026 | |
| (T3) | | | |

TABLE 10
Agonistic activity of compounds toward the thyroxine receptor beta

| | EC₅₀ | |
|---|---|---|
| | THRβ agonistic | THRα agonistic |
| Compound | activity (μM) | activity (μM) |
| 2 | 1.75 | 3.98 |
| 6 | 2.45 | 4.25 |
| 9 | 0.79 | 1.08 |
| 10 | 0.097 | 0.123 |
| Reference Compound | 2.48 | 4.57 |
| triiodothyronine (T3) | 0.001 | 0.0005 |

Compared with the reference compounds, exemplary compounds of formula (II) showed higher THRβ activity (<0.2 μM), and/or higher selectivity to THRα. The data also suggested that the compound of formula (II) can activate the downstream signal of the thyroid hormone receptor beta.

Pharmacokinetic Evaluation: Six healthy male SD rats, commercially available from Shanghai Sippr-Bk Laboratory Animal Co., Ltd., with an animal production license No.: SCXK(Shanghai) 2008-0016, were divided into 2 groups, 3 in each group.

Drug Preparation: a certain amount of the drug was taken and added into a 2% Klucel LF+0.1% Tween 80 aqueous solution, to prepare a clear solution or a uniform suspension.

Dosage: SD rats were fasted overnight and given the drug by intragastric infusion at an administrated dose of 2 mg/kg and an administrated volume of 10 mL/kg each.

Operation: rats were dosed by intragastric infusion with the compounds. At least 0.2 mL of blood was collected from the vena caudalis at 15 min, 30 min, 1 h, 2 h, 4 h, 6 h, 10 h, and 24 h before and after the dosage; the blood was then placed in heparinized sample tubes, centrifuged at 4° C. and 3500 rpm for 10 min to separate the plasma. The heparinized sample tubes were then stored at −20° C., and the rats were allowed to eat food 2 h after the dosage.

Determination of contents of the compounds to be tested in the plasma of rats after intragastric infusion of the drugs at different concentrations: the plasma samples were thawed at room temperature, 50 μL each was taken and added into 130 μL of an internal standard working solution (1000 ng/mL, acetonitrile, tolbutamide), and the mixture was whirled for about 1 min and then centrifugated at 4° C. and 13000 rpm for 10 min 50 μL of the supernatant was taken and mixed with 100 μL of 50% acetonitrile water, and then introduced for LC/MS/MS analysis.

Results of the pharmacokinetic parameters are shown in Table 11.

TABLE 11
Pharmaceutical metabolism data of rats

| | | Time | Peak blood drug | | |
|---|---|---|---|---|---|
| | Dose | to peak | concentration | Curve area | Half-life |
| Compound | (mg/kg) | (h) | (ng/mL) | (ng · h/mL) | (h) |
| 2 | 2.0 | 4.67 ± | 2007 ± 106 | 24790 ± | 4.56 ± |
| | | 1.15 | | 3704 | 0.42 |
| 6 | 2.0 | 5.33 ± | 727 ± 183 | 9242 ± | 5.14 ± |
| | | 1.15 | | 1245 | 0.83 |
| Reference | 2.0 | 5.3 ± | 1163 ± 97.1 | 12854 ± | 3.53 ± |
| Compound | | 1.15 | | 961 | 0.42 |

The data showed that exemplary compounds demonstrated good pharmacokinetic absorption and significant pharmacokinetic advantages. Compared with the reference compound, exemplary compounds showed higher C max values and exposure amounts at the same dose and preparation.

### Example 11: Effects on Serum Cholesterol and Triglycerides

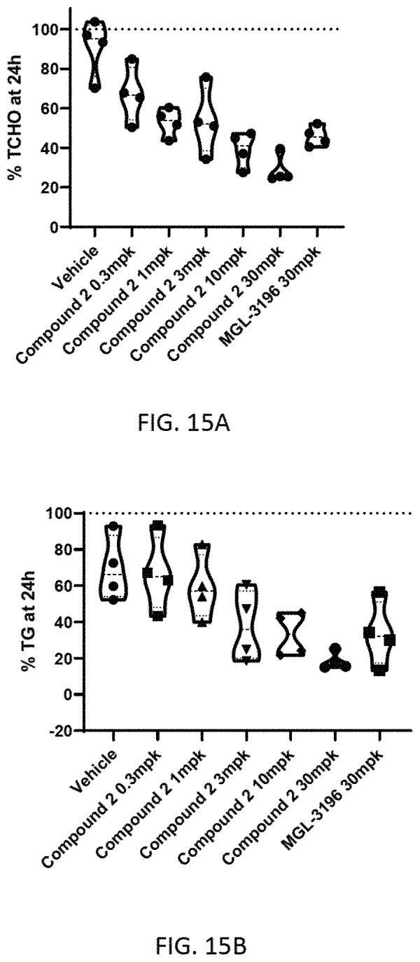

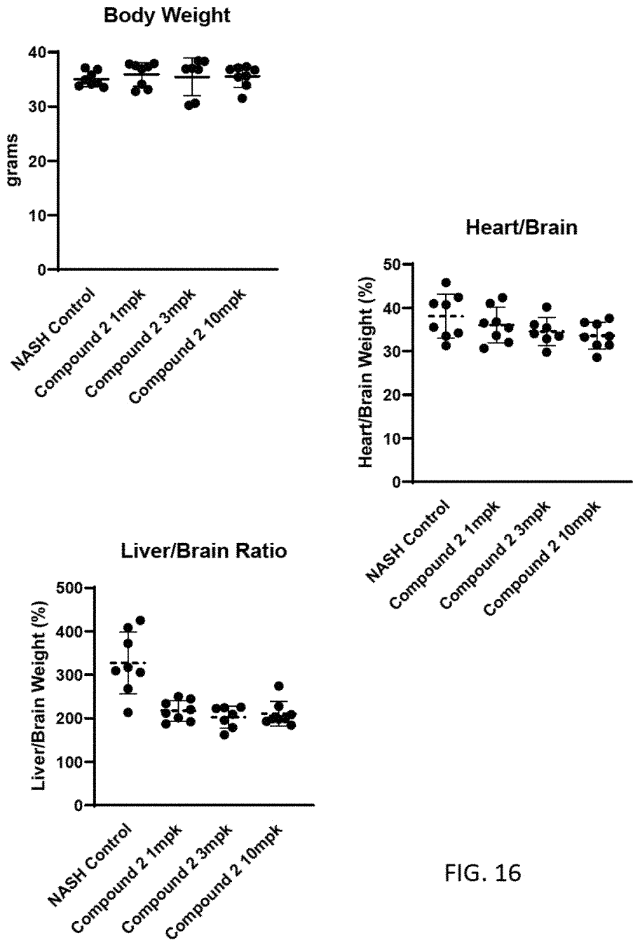

SD rats were fed a high cholesterol diet for 2 weeks, increasing the serum cholesterol levels ˜4-fold over that time. Single doses of Compound 2 from 0.3 to 30 mpk or a single 30 mpk dose of MGL-3196 were injected IP and serum was analyzed for total serum cholesterol and triglycerides 24 h after the injection. Total cholesterol in the serum was significantly reduced from 30-70% with Compound 2 (FIG. 15A). Compound 2 significantly reduced serum triglycerides from 30-80% from time 0 (FIG. 15B).

### Example 12: Effects on Mouse NASH Model

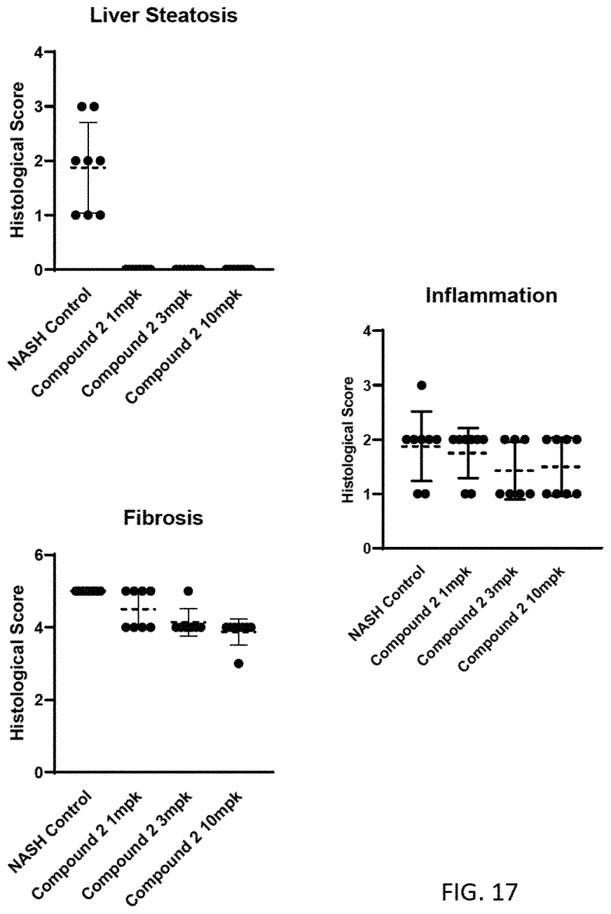

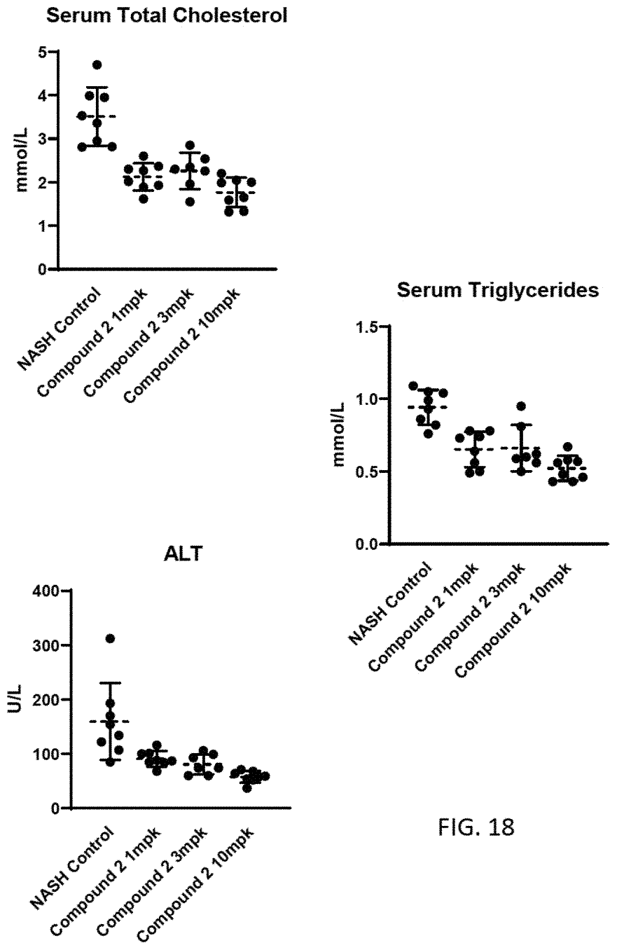

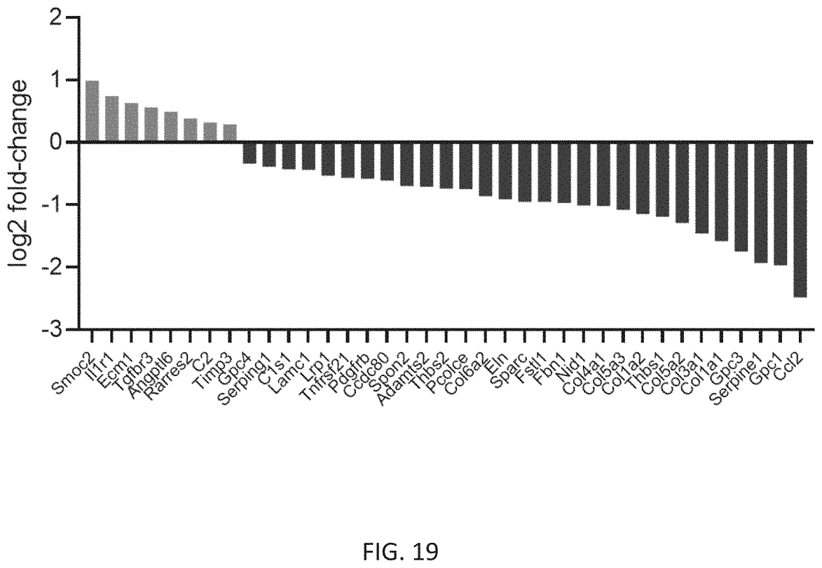

C57BL/6J mice were fed a high fat diet for 10 weeks to induce obesity (>38 g BW). Obese mice were injected intraperitoneally (i.p.) twice a week for four weeks with 0.5 μl/g 25% CCl₄ (formulated in olive oil) to induce fibrosis, and one group of normal BW mice were injected i.p. twice a week for four weeks with olive oil to serve as a healthy control. During the same dosing period, obese mice were fed orally once a day for 28 days with vehicle or varying doses of Compound 2. On CCl₄ dosing days, CCl₄ was administered at 4 hours post compound or vehicle dosing. On day 27, all animals were fasted for about 16 hours before terminal euthanasia. On day 28, all animals were sacrificed and various biological parameters were analyzed. Total body, liver, heart and brain weight were measured and changes in liver and heart weight were normalized using brain weight. Compound 2 significantly reduced liver/brain weight with no effect on total body weight or heart/brain weight (FIG. 16). Liver tissue histology was analyzed for effects of Compound 2 on steatosis, inflammation and fibrosis. Compound 2 significantly reduced steatosis at all doses tested, showed a trend in inflammation reduction and significantly reduced liver fibrosis at 3 and 10 mpk (FIG. 17). Compound 2 also significantly reduced serum total cholesterol, triglycerides and ALT at all doses tested (FIG. 18). Liver samples were collected for whole transcriptome analysis by RNA sequencing (RNAseq). RNAseq library (n=5 per group) preparation and sequencing was performed using Illumina standard protocols. Alignment of sequencing reads was performed using STAR aligner software and read counts were estimated using RSEM. Differentially expressed genes (compared to vehicle-treated NASH control mice) were determined using EdgeR software. Gene ontology analysis was performed using Advaita software with fold-change and adjusted p-value cutoffs of >1.5 and <0.05, respectively. Gene ontologies were derived from the Gene Ontology Consortium database (2019 Apr. 26) (Ashburner et al., Gene ontology: Tool for the unification of biology. Nature Genetics 25(1): 25-9 (2000); Gene Ontology Consortium, Creating the Gene Ontology Resource: Design and Implementation. Genome Research 11: 1425-1433 (2001)). Compound 2 had a significant effect on expression of genes associated with collagen extracellular matrix and hepatic stellate cell activation, primarily by reducing their expression levels relative to NASH control mice (FIG. 19).

### Example 13: Differentially Expressed Genes (DEGs)

C57BL/6J mice were fed a high fat diet for 10 weeks to induce obesity (>38 g BW). Obese mice were injected intraperitoneally (i.p.) twice a week for four weeks with 0.5 μl/g 25% CCl₄ (formulated in olive oil) to induce fibrosis, and one group of normal BW mice were injected i.p. twice a week for four weeks with olive oil to serve as a healthy control. During the same dosing period, obese mice were fed orally once a day for 28 days with vehicle, Compound 1 or Compound 2 as single agents or in combination. On CCl₄ dosing days, CCl₄ was administered at 4 hours post compound or vehicle dosing. On day 27, all animals were fasted for about 16 hours before terminal euthanasia. On day 28, all animals were sacrificed and liver samples were collected for whole transcriptome analysis by RNA sequencing (RNAseq). RNAseq library (n=5 per group) preparation and sequencing was performed using Illumina standard protocols. Alignment of sequencing reads was performed using STAR aligner software and read counts were estimated using RSEM. Differentially expressed genes (compared to vehicle-treated NASH control mice) were determined using EdgeR software. Gene ontology analysis was performed using Advaita software with fold-change and adjusted p-value cutoffs of >1.5 and <0.05, respectively. Gene ontologies were derived from the Gene Ontology Consortium database (2019 Apr. 26) (Ashburner et al., Gene ontology: Tool for the unification of biology. Nature Genetics 25(1): 25-9 (2000); Gene Ontology Consortium, Creating the Gene Ontology Resource: Design and Implementation. Genome Research 11: 1425-1433 (2001)).

The change direction (i.e., up or down) and total number of differentially expressed genes (DEGs) identified between vehicle-treated NASH controls and mice treated with Compound 1 (3 mg/mg), Compound 2 (1 mg/kg), or the combination of Compound 1 (3 mg/kg) and Compound 2 (1 mg/kg) are shown in Table 12. Using an absolute fold-change cutoff of >1.5-fold and adjusted p-value of <0.05, 617 DEGs were identified in Compound 1 treated mice, 1113 DEGs were identified in Compound 2 treated mice, and 1871 DEGs were identified in mice treated with the combination of Compound 1 and Compound 2. These results suggest that the combination treatment resulted in at least additive effects on the total number of DEGs relative to the arithmetic sum of DEGs identified from each single treatment group. The number of down regulated DEGs (Down DEGs) was higher in the combination treatment group compared to the arithmetic sum of Down DEGs from each single agent treatment group. These results indicated that the combination of Compound 1 and Compound 2 resulted in a larger than expected number of DEGs relative to single agent treatments and this effect was the result of a larger than expected number of down regulated DEGs.

TABLE 12
Differentially expressed genes (DEGs)

| | Down | Up | Total |
|---|---|---|---|
| Treatment group | DEGs | DEGs | DEGs |
| Compound 1 (3 mg/kg) | 271 | 346 | 617 |
| Compound 2 (1 mg/kg) | 635 | 478 | 1113 |
| Compound 1 (3 mg/kg) + | 1182 | 689 | 1871 |
| Compound 2 (1 | | | |
| mg/kg) | | | |

Number of DEGs identified (vehicle NASH control vs. treatment) identified for each treatment group. Adjusted p value<0.05 and fold-change >1.5-fold

### Example 14: Gene Ontology (GO) Enrichment Analysis

Gene ontology (GO) enrichment analysis was used to understand the potential biological consequences of the results in Table 12. To perform GO term enrichment analysis, the number (i.e., enrichment) of DEGs annotated for a particular term (i.e. biology process) was compared to the number of DEGs expected solely by chance. An over-representation approach was used to compute statistical significance (p-value) of observing at least the given number of DEGs; p-values reported in Table 6 were corrected for multiple comparisons.

Liver inflammation is a defining characteristic and key driver of NASH disease and is mediated in large part by overactivation and infiltration of leukocytes into the liver. Therapies that target inflammatory processes directly via anti-inflammatory mechanisms or indirectly by, for example, decreasing oxidative stress by normalizing metabolic function and reducing liver steatosis, have the potential to impact NASH disease. Table 13 shows GO term enrichment analysis for DEGs associated with leukocyte-related biological processes. As shown in Table 13, only the combination of Compound 1 and Compound 2 showed a statistically significant enrichment of DEGs associated with leukocyte-related biological processes. These results suggested that the combination of Compound 1 with Compound 2 had a much more profound effect on leukocyte-related biological processes than either single treatment alone.

TABLE 13
GO term enrichment analysis for leukocyte-related biological processes

| | | Compound 1 | Compound 2 | Compound 1 + |
|---|---|---|---|---|
| Biological process | GO ID | (3 mg/kg) | (1 mg/kg) | Compound 2 |
| myeloid leukocyte activation | GO: 0002274 | 0.52 | 0.36 | 1.6E−08 |
| leukocyte activation | GO: 0045321 | 0.73 | 0.45 | 5.8E−08 |
| leukocyte migration | GO: 0050900 | 0.47 | 0.36 | 2.3E−07 |
| leukocyte activation involved in | GO: 0002269 | 0.38 | 0.1 | 5.1E−06 |
| inflammatory response | | | | |
| myeloid leukocyte migration | GO: 0097529 | 0.74 | 0.52 | 1.1E−05 |
| leukocyte chemotaxis | GO: 0030595 | 0.65 | 0.45 | 2.6E−05 |
| leukocyte cell-cell adhesion | GO: 0007159 | 0.58 | 0.36 | 6.9E−05 |
| leukocyte proliferation | GO: 0070661 | 0.79 | 0.62 | 9.4E−05 |
| regulation of leukocyte migration | GO: 0002685 | 0.49 | 0.25 | 0.00017 |
| leukocyte mediated immunity | GO: 0002443 | 0.71 | 0.84 | 0.00018 |

Adjusted p-values shown for each treatment group. Top ten leukocyte-associated biological processes enriched in the Compound 1 and Compound 2 combination treatment group shown.

Table 14 shows GO term enrichment analysis for DEGs associated with immune and leukocyte-related biological processes that were uniquely enriched by combination treatment as described in Example 13.

TABLE 14
GO term enrichment analysis of immune-related biological pathways
uniquely enriched by combination treatment

| | | DEG | Total Genes | Corrected |
|---|---|---|---|---|
| Biological process | GO term ID | count (n) | (n) | p-value |
| immune response | GO: 0006955 | 216 | 941 | 1.21E−10 |
| inflammatory response | GO: 0006954 | 124 | 467 | 1.12E−09 |
| myeloid leukocyte activation | GO: 0002274 | 55 | 156 | 1.59E−08 |
| immune system process | GO: 0002376 | 327 | 1674 | 3.94E−08 |
| leukocyte activation | GO: 0045321 | 145 | 615 | 5.79E−08 |
| positive regulation of immune | GO: 0002684 | 156 | 687 | 1.86E−07 |
| system process | | | | |
| leukocyte migration | GO: 0050900 | 69 | 233 | 2.33E−07 |
| regulation of immune response | GO: 0050776 | 132 | 567 | 5.75E−07 |
| regulation of immune system | GO: 0002682 | 202 | 972 | 9.68E−07 |
| process | | | | |
| leukocyte activation involved in | GO: 0002269 | 18 | 32 | 5.1E−06 |
| inflammatory response | | | | |
| myeloid leukocyte migration | GO: 0097529 | 45 | 142 | 1.09E−05 |
| leukocyte chemotaxis | GO: 0030595 | 44 | 142 | 2.6E−05 |
| positive regulation of immune | GO: 0050778 | 104 | 455 | 3.94E−05 |
| response | | | | |
| innate immune response | GO: 0045087 | 113 | 508 | 5.06E−05 |
| leukocyte cell-cell adhesion | GO: 0007159 | 61 | 231 | 6.9E−05 |
| leukocyte proliferation | GO: 0070661 | 59 | 223 | 9.41E−05 |
| neuroinflammatory response | GO: 0150076 | 20 | 47 | 0.000173 |
| regulation of leukocyte migration | GO: 0002685 | 43 | 148 | 0.000173 |
| leukocyte mediated immunity | GO: 0002443 | 66 | 265 | 0.000185 |
| cell activation involved in immune | GO: 0002263 | 51 | 192 | 0.000323 |
| response | | | | |
| leukocyte activation involved in | GO: 0002366 | 50 | 188 | 0.000373 |
| immune response | | | | |
| regulation of leukocyte activation | GO: 0002694 | 87 | 386 | 0.000383 |
| regulation of inflammatory | GO: 0050727 | 63 | 256 | 0.000397 |
| response | | | | |
| positive regulation of leukocyte | GO: 0002696 | 58 | 230 | 0.000406 |
| activation | | | | |
| adaptive immune response | GO: 0002250 | 69 | 293 | 0.000639 |
| positive regulation of leukocyte | GO: 0002687 | 32 | 106 | 0.000913 |
| migration | | | | |
| immune effector process | GO: 0002252 | 114 | 554 | 0.001036 |
| positive regulation of inflammatory | GO: 0050729 | 29 | 93 | 0.001062 |
| response | | | | |
| neutrophil activation involved in | | 9 | 14 | 0.001102 |
| immune response | | | | |
| immune response-activating signal | GO: 0002757 | 59 | 246 | 0.001269 |
| transduction | | | | |
| regulation of leukocyte | GO: 0070663 | 44 | 168 | 0.001381 |
| proliferation | | | | |
| immune response-regulating | GO: 0002764 | 60 | 255 | 0.001816 |
| signaling pathway | | | | |
| leukocyte aggregation | GO: 0070486 | 8 | 12 | 0.001944 |
| regulation of leukocyte mediated | GO: 0002703 | 42 | 164 | 0.003108 |
| immunity | | | | |
| positive regulation of immune | GO: 0002699 | 43 | 170 | 0.003403 |
| effector process | | | | |
| positive regulation of leukocyte | GO: 1903039 | 38 | 145 | 0.003827 |
| cell-cell adhesion | | | | |
| regulation of leukocyte cell-cell | GO: 1903037 | 50 | 208 | 0.003827 |
| adhesion | | | | |
| myeloid cell activation involved in | GO: 0002275 | 21 | 64 | 0.004329 |
| immune response | | | | |
| positive regulation of leukocyte | GO: 0002690 | 21 | 64 | 0.004329 |
| chemotaxis | | | | |
| leukocyte differentiation | GO: 0002521 | 86 | 413 | 0.004907 |
| activation of immune response | GO: 0002253 | 68 | 312 | 0.005594 |
| myeloid leukocyte mediated | GO: 0002444 | 22 | 70 | 0.005847 |
| immunity | | | | |
| positive regulation of leukocyte | GO: 0002705 | 29 | 104 | 0.006575 |
| mediated immunity | | | | |
| acute inflammatory response | GO: 0002526 | 24 | 81 | 0.007746 |
| leukocyte degranulation | GO: 0043299 | 17 | 50 | 0.00959 |
| regulation of leukocyte chemotaxis | GO: 0002688 | 23 | 78 | 0.010243 |
| immune response-activating cell | GO: 0002429 | 35 | 140 | 0.012131 |
| surface receptor signaling pathway | | | | |
| regulation of myeloid leukocyte | GO: 0002886 | 16 | 47 | 0.012275 |
| mediated immunity | | | | |
| immune response-regulating cell | GO: 0002768 | 36 | 147 | 0.014639 |
| surface receptor signaling pathway | | | | |
| positive regulation of leukocyte | GO: 0070665 | 26 | 99 | 0.023913 |
| proliferation | | | | |

Top 50 immune-related biological processes that were uniquely enriched by Compound 1 (3 mg/kg) and Compound 2 (1 mg/kg) combination treatment. The number of enriched DEGs, total number of genes comprising the biological process, and adjusted p-values are shown.

### Example 15: Differential Gene Expression Analysis of Select Biological Processes

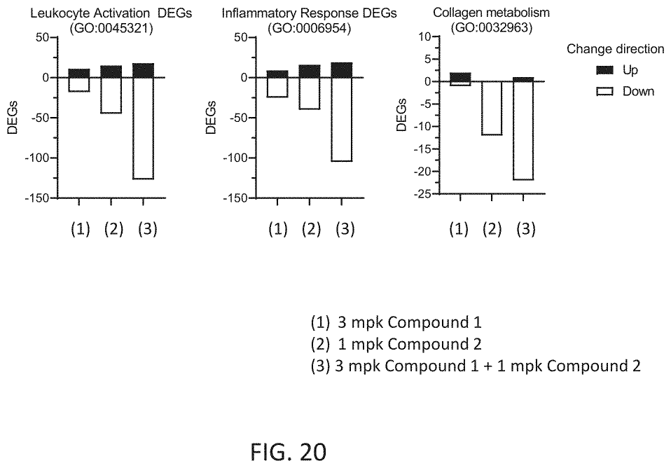

Other biological processes relevant to NASH disease were also examined. FIG. 20 shows the number of Up and Down regulated DEGs (vehicle NASH control vs. treatment) associated with different biological processes relevant to NASH and fibrosis including: leukocyte activation (GO:0045321); inflammatory response (GO:0006954), and collagen metabolic process (GO:0032963). For each biological process examined, the combination of Compound 1 with Compound 2 consistently showed greater than expected number of DEGs relative to single agent treatment groups. In addition, the combination of Compound 1 with Compound 2 showed a greater than expected number of down regulated DEGs than would have been expected based on the results of single agent treatment.

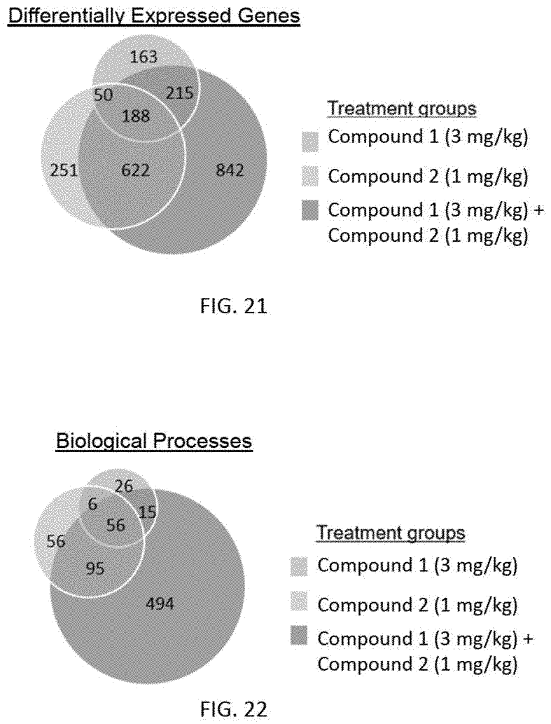

FIG. 21 shows the number and overlap of DEGs (vs. vehicle NASH control) identified in each treatment group using absolute fold-change and adjusted p-value cutoffs of ≥1.5 and <0.05, respectively. The total number of differentially expressed genes was greater than expected with Compound 1 and Compound 2 in combination, with >800 unique to the combination, and this was largely driven by a higher number of downregulated DEGs. FIG. 22 shows the number and overlap of biological processes that were significantly enriched in treatment groups relative to NASH control. An FUR-adjusted p-value of <0.05 was used as a cut-off for statistical significance.

### Example 16: Additional Effects on Mouse NASH Model

On day 28 of treatment as described in Example 13, animals were euthanized for sample collections. Analysis of cholesterol, triglycerides, and ALT was done using a Hitachi 7180 clinical analyzer. Liver samples were processed for lipid quantification (colorimetric assays, SpectraMax 340PC384), histology, and RNA analysis. RNAseq library preparation (n=5 per group) and sequencing was performed using Illumina standard protocols. Alignment of sequencing reads was performed using STAR aligner and read counts were estimated using RSEM. Differentially expressed genes (dEGs) relative to NASH control were determined using EdgeR. Gene ontology analysis was performed using Advaita software.

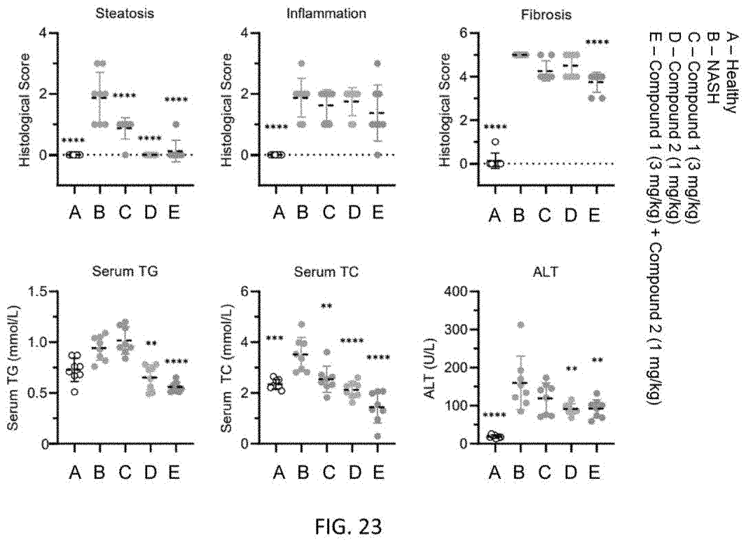

FIG. 23 shows liver steatosis, inflammation, and fibrosis as quantified by histological analysis for degree of steatosis, lobular inflammation, and fibrosis. Serum was collected at termination and analyzed for triglycerides (TG), total cholesterol (TC), and a biomarker of liver damage, alanine aminotransferase (ALT). Data for individual animals (dots) and mean (dashed line) are presented; ** p<0.01, *** p<0.001, **** p<0.0001 vs NASH vehicle control (NASH). Statistics determined by one-way ANOVA followed by Tukey. The combination treatment of Compound 1 and Compound 2 significantly improved multiple components of NASH, including steatosis, fibrosis, serum triglycerides, total cholesterol, and liver damage as measured by ALT.

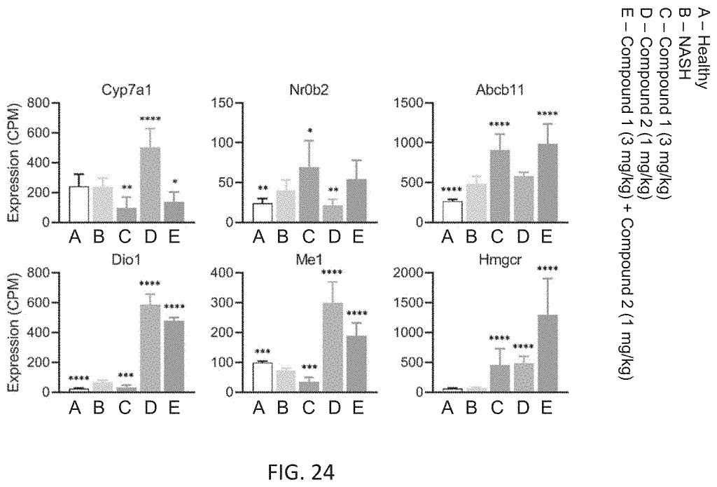

FIG. 24 shows mean expression levels of genes associated with FXR and THRβ pathway activation. FXR and THRβ pathway genes were modulated in both single and combination treatment groups.

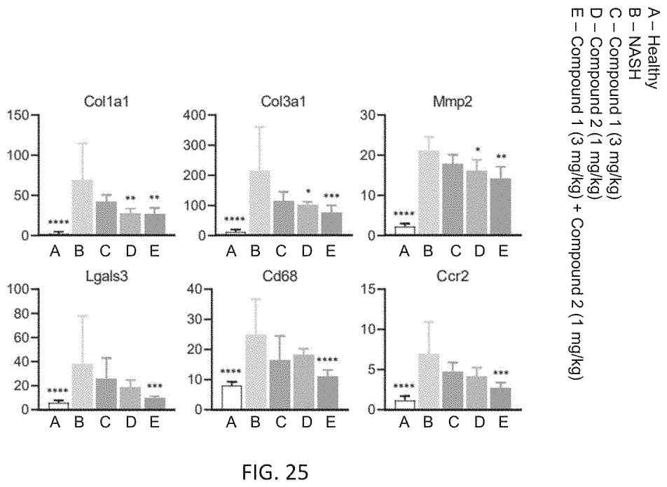

FIG. 25 shows mean expression levels (count per million reads, CPM) of genes associated with collagen/fibrosis and inflammation pathways, which were determined by RNAseq. *p<0.05, **p<0.01, ***p<0.001, ****p<0.0001 vs. vehicle (NASH) control. Error bars represent standard deviation (n=5). The combination treatment of Compound 1 and Compound 2 significantly reduced expression of collagen/fibrosis genes and inflammatory genes such as Col1a1, Col3a1, Mmp2, Lgals3, Cd68, and Ccr2.

Conclusions

Treatment with Compound 1 and Compound 2 in combination resulted in gene expression changes that were consistent with on-target agonism of FXR and THRβ, respectively. The combination treatment of Compound 1 and Compound 2 significantly reduced expression of fibrosis and inflammatory genes.

Gene ontology enrichment analysis identified the unpredictable result that nearly 500 biological processes were uniquely enriched by Compound 1 and Compound 2 combination treatment, including down-regulation of those related to immune processes (inflammation), leukocyte function, and collagen (including collagen production) (see FIG. 20, FIG. 25). Together these data support the concept that the combination of Compound 1 and Compound 2 may provide additional benefit in NASH relative to single agent therapies, such as reducing the inflammatory component or fibrotic component of NASH more significantly than a single agent therapy alone. These affects are expected to reduce disease severity, as well as disease progression.

### Example 17: Safety, Tolerability, Efficacy of Combination Therapy in Patients with NASH

A randomized, double-blind, placebo-controlled study is conducted to evaluate the safety and efficacy of combination treatments, for example, Compound 1 and Compound 2. Subjects with NASH are treated once daily with the FXR agonist and the THRβ agonist in combination for 12 or 48 weeks. Liver fat is monitored by MRI-PDFF, and serum-based non-invasive fibrosis or NASH markers such as C3, TIMP-1, PIIINP, CK-18, and ALT, are measured. Side effects such as pruritus and LDL-C cholesterol levels are also monitored.

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

1. A method of treating non-alcoholic steatohepatitis (NASH) in a patient in need thereof, comprising administering to the patient a Farnesoid X Receptor (FXR) agonist and a THRβ agonist, wherein the FXR agonist is a compound of formula (1): 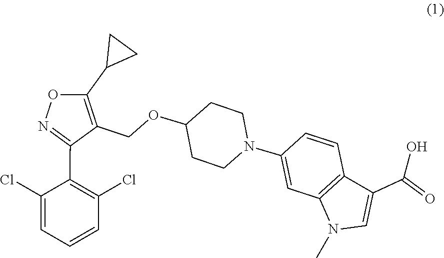 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof.

2-9. (canceled)

10. The method of claim 1, wherein the THRβ agonist is a compound of formula (II) 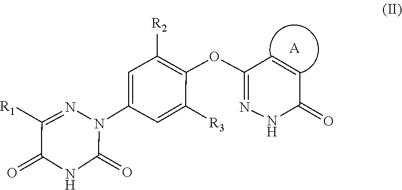
wherein:
R₁ is selected from the group consisting of hydrogen, cyano, substituted or unsubstituted C₁₋₆ alkyl, and substituted or unsubstituted C₃₋₆ cycloalkyl, the substituent being selected from the group consisting of halogen atoms, hydroxy, and C₁₋₆ alkoxy;
R₂ and R₃ are each independently selected from the group consisting of halogen atoms and substituted or unsubstituted C₁₋₆ alkyl, the substituent being selected from the group consisting of halogen atoms, hydroxy, and C₁₋₆ alkoxy;
ring A is a substituted or unsubstituted saturated or unsaturated C₅₋₁₀ aliphatic ring, or a substituted or unsubstituted C₅₋₁₀ aromatic ring, the substituent being one or more substances selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl, and when two substituents are contained, the two substituents can form a ring structure together with the carbon connected thereto; and
the halogen atoms are selected from the group consisting of F, Cl and Br, or a pharmaceutically acceptable salt thereof.

11. The method of claim 10, wherein the THRβ agonist is a compound of formula (IIa) 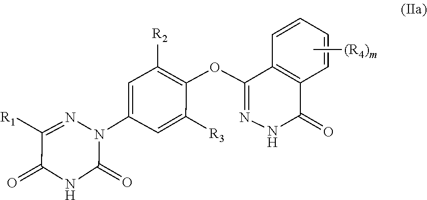
wherein:
R₁ to R₃ are defined as described in claim 10;
R₄ is selected from the group consisting of hydrogen, halogen atoms, hydroxy, —OCF₃, —NH₂, —NHC₁₋₄ alkyl, —N(C₁₋₄ alkyl)₂, —CONH₂, —CONHC₁₋₄ alkyl, —CON(C₁₋₄ alkyl)₂, —NHCOC₁₋₄ alkyl, C₁₋₆ alkyl, C₁₋₆ alkoxy and C₃₋₆ cycloalkyl;
m is an integer from the range 1 to 4; and
the halogen atoms are selected from the group consisting of F, Cl and Br. or a pharmaceutically acceptable salt thereof.

12. The method of claim 10, wherein R₄ is selected from the group consisting of hydrogen, halogen atoms, hydroxy, —OCF₃, C₁₋₆ alkyl, C₁₋₆ alkoxy and C₃₋₆ cycloalkyl; and
m is an integer from the range 1 to 3.

13. The method of claim 10, wherein R₁ is selected from the group consisting of hydrogen, cyano, and substituted or unsubstituted C₁₋₆ alkyl, the substituent being selected from the group consisting of halogen atoms, hydroxy, and C₁₋₆ alkoxy; and
the halogen atoms are selected from the group consisting of F, Cl and Br.

14. The method of claim 1, wherein the THRβ agonist is a compound of formula (2): 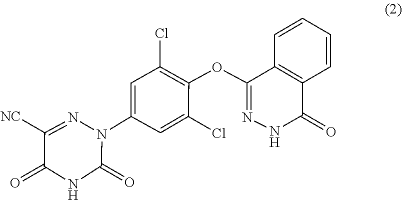 [SMILES: N#Cc1nn(-c2cc(Cl)c(Oc3n[nH]c(=O)c4ccccc34)c(Cl)c2)c(=O)[nH]c1=O]
or a pharmaceutically acceptable salt thereof.

15. The method of claim 14, wherein the FXR agonist and the THRβ agonist are administered simultaneously.

16. The method of claim 14, wherein the FXR agonist and the THRβ agonist are administered sequentially.

17. The method of claim 1, wherein the administration does not result in pruritus in the patient at a severity of Grade 2 or more.

18. The method of claim 1, wherein the administration does not result in pruritus in the patient at a severity of Grade 1 or more.

19. The method of claim 1, wherein the patient also has a cardiovascular disorder.

20. The method of claim 1, wherein the patient also has diabetes mellitus.

21-41. (canceled)

42. A method of reducing hepatic inflammation in a patient in need thereof, comprising administering to the patient a therapeutically effective amount of a FXR agonist and a therapeutically effective amount of a THRβ agonist, wherein the FXR agonist is a compound of formula (1): 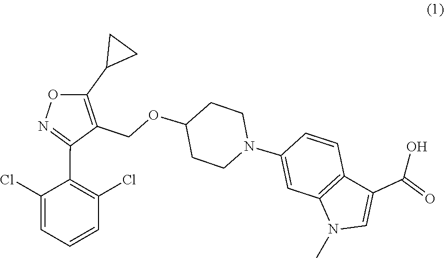 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof, and the THRβ agonist is a compound of formula (2): 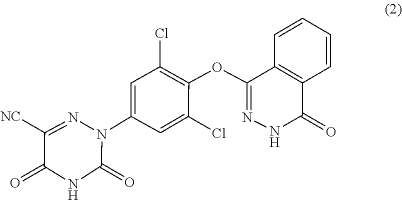 [SMILES: N#Cc1nn(-c2cc(Cl)c(Oc3n[nH]c(=O)c4ccccc34)c(Cl)c2)c(=O)[nH]c1=O]
or a pharmaceutically acceptable salt thereof.

43. A method of reducing hepatic inflammation in a patient in need thereof without increasing LDL-C levels in the patient, said method comprising administering to the patient a therapeutically effective amount of a FXR agonist and a therapeutically effective amount THRβ agonist, wherein the FXR agonist is a compound of formula (1): 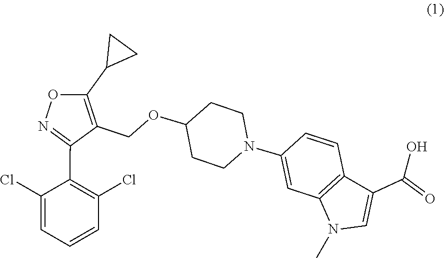 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof, and the THRβ agonist is a compound of formula (2): 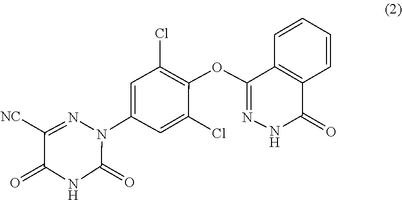 [SMILES: N#Cc1nn(-c2cc(Cl)c(Oc3n[nH]c(=O)c4ccccc34)c(Cl)c2)c(=O)[nH]c1=O]
or a pharmaceutically acceptable salt thereof.

44-47. (canceled)

48. The method of claim 14, wherein the patient has liver fibrosis.

49-52. (canceled)

53. The method of claim 14, wherein the THRβ agonist is administered at a dose that reduces LDL-C levels in the patient.

54. The method of claim 14, wherein the THRβ agonist is administered at a dose that prevents an increase in LDL-C levels in the patient.

55-83. (canceled)

84. The method of claim 14, wherein the FXR agonist is a compound of formula (1):

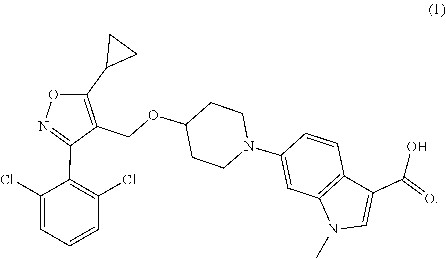 [SMILES: Cn1cc(C(=O)O)c2ccc(N3CCC(OCc4c(-c5c(Cl)cccc5Cl)noc4C4CC4)CC3)cc21]

85. The method of claim 14, wherein the THRβ agonist is a potassium salt of the compound of formula (2).

86. The method of claim 84, wherein the THRβ agonist is a potassium salt of the compound of formula (2).

84. The method of claim 14, wherein the compound of formula (1), or a pharmaceutically salt thereof, is administered to the patient at a dose from about 1 mg to about 15 mg daily and the compound of formula (2), or a pharmaceutically salt thereof, is administered to the patient at a dose from about 3 mg to about 90 mg daily.

85. The method of claim 84, wherein the compound of formula (1), or a pharmaceutically salt thereof, and the compound of formula (2), or a pharmaceutically salt thereof, are each administered once daily to the patient.

86. The method of claim 84, wherein the FXR agonist is a compound of formula (1): 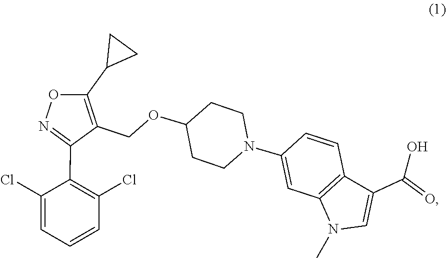 [SMILES: Cn1cc(C(=O)O)c2ccc(N3CCC(OCc4c(-c5c(Cl)cccc5Cl)noc4C4CC4)CC3)cc21]
and the THRβ agonist is a potassium salt of the compound of formula (2).

87. The method of claim 14, wherein the compound of formula (1), or a pharmaceutically salt thereof, is administered to the patient at a dose from about 5 mg to about 15 mg daily and the compound of formula (2), or a pharmaceutically salt thereof, is administered to the patient at a dose from about 0.5 mg to about 30 mg daily.

88. The method of claim 14, wherein the compound of formula (1), or a pharmaceutically salt thereof, is administered to the patient at a dose from about 1 mg to about 10 mg daily and the compound of formula (2), or a pharmaceutically salt thereof, is administered to the patient at a dose from about 0.5 mg to about 30 mg daily.

89. The method of claim 88, wherein the FXR agonist is a compound of formula (1): 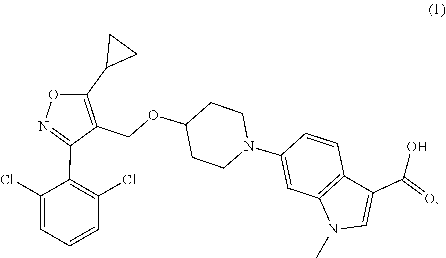 [SMILES: Cn1cc(C(=O)O)c2ccc(N3CCC(OCc4c(-c5c(Cl)cccc5Cl)noc4C4CC4)CC3)cc21]
and the THRβ agonist is a potassium salt of the compound of formula (2).

90. A fixed-dose pharmaceutical composition for oral administration, comprising a compound of formula (1): 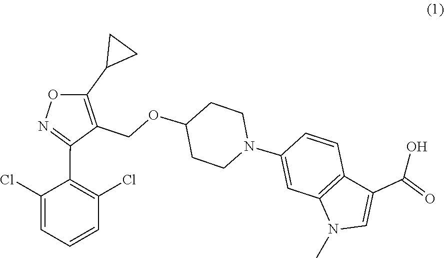 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof, and a compound of formula (2): 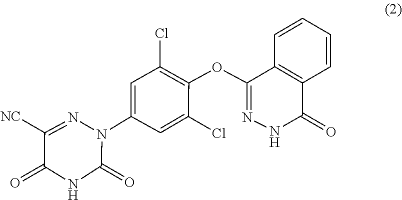 [SMILES: N#Cc1nn(-c2cc(Cl)c(Oc3n[nH]c(=O)c4ccccc34)c(Cl)c2)c(=O)[nH]c1=O]
or a pharmaceutically acceptable salt thereof.

91. The fixed-dose pharmaceutical composition of claim 90, wherein the composition comprises from about 5 mg to about 15 mg of the compound of formula (1), or a pharmaceutically salt thereof, and from about 0.5 mg to about 30 mg of the compound of formula (2), or a pharmaceutically salt thereof.

92. The fixed-dose pharmaceutical composition of claim 90, wherein the composition comprises from about 5 mg to about 15 mg of the compound of formula (1), or a pharmaceutically salt thereof, and from about 20 mg to about 50 mg of the compound of formula (2), or a pharmaceutically salt thereof.

93. The fixed-dose pharmaceutical composition of claim 91, wherein the FXR agonist is a compound of formula (1): 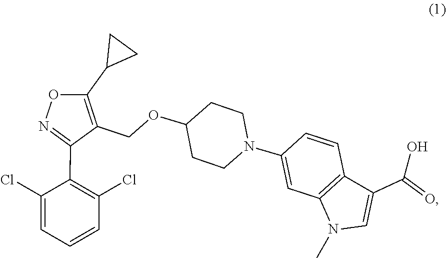 [SMILES: Cn1cc(C(=O)O)c2ccc(N3CCC(OCc4c(-c5c(Cl)cccc5Cl)noc4C4CC4)CC3)cc21]
and the THRβ agonist is a potassium salt of the compound of formula (2).
